(19) United States
(12) Patent Application Publication
(10) Pub. No.: US 2022/0401627 A1
(43) Pub. Date: Dec. 22, 2022

(54) TISSUE REPAIR IMPLANT AND COMPOSITIONS AND METHOD OF IMPLANTATION

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: George Barnes, Natick, MA (US); Hans P.L. Claesson, Lexington, MA (US); Timothy Young, Natick, MA (US); Nicholas J. Cotton, Westborough, MA (US); Paul A. Torrie, Marblehead, MA (US); Craig L. Van Kampen, Oakdale, MN (US); David A. Yeung, Andover, MA (US); Cori G. Pierce, Salem, NH (US); Jeffrey L. Barnes, Medford, MA (US); Jessica M. Grabinsky, Melrose, MA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 17/897,789

(22) Filed: Aug. 29, 2022

Related U.S. Application Data
(63) Continuation of application No. PCT/US2021/021935, filed on Mar. 11, 2021.
(60) Provisional application No. 62/988,635, filed on Mar. 12, 2020.
(60) Provisional application No. 63/039,481, filed on Jun. 16, 2020.

(51) Int. Cl. A61L 27/56; A61L 27/58; A61L 27/26

(52) U.S. Cl. CPC A61L 27/56; A61L 27/58; A61L 27/26; A61L 2430/10

(57) ABSTRACT

A tendon/ligament repair implant for treatment of tears or lesions of tendons and ligaments, including capsular reconstruction, and compositions for delivering calcium and/or phosphate ions in combination with a collagen solution that can be placed between soft tissue and bone to facilitate healing of the soft tissue-bone interface are provided. The implant may incorporate features of rapid deployment and fixation by arthroscopic means that complement current procedures; tensile properties that result in desired sharing of anatomical load between the implant and native tendon during rehabilitation, or, in situations where the native tissue cannot be repaired tensile properties that provide for substitution of the native tissue selected porosity and longitudinal pathways for tissue in-growth; and may include an at least partially bioabsorbable construction to provide transfer of additional load to new tendon-like tissue and native tendon over time. The compositions can be pre-dried into a thin sheet of material and delivered as a pre-formed matrix, or as a gel or paste which sets in place to form the matrix between the soft tissue and bone.

### CROSS REFERENCE TO RELATED APPLICATIONS

This application is a continuation of International Application No. PCT/US2021/021935, filed Mar. 11, 2021, titled TISSUE REPAIR IMPLANT AND COMPOSITIONS AND METHOD OF IMPLANTATION, which claims the benefit of and priority to U.S. Provisional Patent Application Ser. No. 62/988,635, filed on Mar. 12, 2020, titled TISSUE REPAIR IMPLANT AND METHOD OF IMPLANTATION, and to U.S. Provisional Patent Application Ser. No. 63/039,481, filed on Jun. 16, 2020, titled COMPOSITIONS AND METHODS FOR SOFT TISSUE-TO-BONE REPAIR, the disclosures of which are incorporated herein by reference.

### TECHNICAL FIELD

The present disclosure pertains generally, but not by way of limitation, to orthopedic implants, compositions, and methods of treatment. More particularly, the present invention relates to a tendon/ligament repair implant, such as one that is engineered for placement in the area of a tear or lesion of a tendon or ligament, and compositions and methods for promoting and enhancing healing at a soft tissue-to-bone interface in a surgical repair.

### BACKGROUND

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. Large to massive rotator cuff tears remain a challenge for surgeons, particularly when the tissue to be repaired or re-approximated to its natural location is degenerated. Patient age, tear size, and tendon retraction all contribute to the difficulty of repair. Outcomes may be poor with a re-tear rate of between about 40% to about 90%.

Biological tissue scaffolds (e.g., autografts, allografts, xenografts, etc.) may be used to reinforce degenerated tendons in these challenging repairs. Autografts require sacrifice of functional tissue and usually allografts or xenografts may be the preferred option. These biological tissue scaffolds can mostly support natural tissue ingrowth and replacement unless the scaffold is too dense or overly chemically altered. Allograft and/or xenograft scaffolds may be convenient (e.g., off the shelf) but may be limited as they do not match the mechanical properties of the natural tendon and are therefore not indicated for critical “bridging” indications where the tendon is severely retracted and a gap needs to be bridged to the bone.

Some rotator cuff tears are irreparable. An alternative technique is the superior capsular reconstruction (SCR) which utilizes the biological tissue scaffolds noted above, to stabilize the joint by direct attachment of the glenoid to the humeral head. Again allograft and xenograft scaffolds may be utilized for this technique as they are convenient but again they do not possess appropriate mechanical properties. Allograft and/or xenograft scaffolds are usually attached and fixed using conventional sutures and this fixation is usually the weak point of the repair as the high strength synthetic sutures may rip through the biological tissue. Synthetic scaffolds have been utilized for both rotator cuff repair and superior capsule reconstruction but are not widely accepted and more biological acceptable materials are preferred.

Other tendons or ligaments in the body, such as, but not limited to the hip capsule, may also be difficult to repair. The hip capsule is a high strength tissue in the human body which encapsulates the hip ball and socket joint space. It is in part high strength due to the forces it withstands during various ranges of motion of the hip. The hip capsule is routinely cut in order to perform arthroscopic hip surgery. At the end of hip arthroscopy, it is desirable to repair the capsule. While the use of a xenograft (or other) scaffold may facilitate healing of the tissue, a scaffold may be a non-structural implant that is susceptible to tearing under loading. In some cases, there may not be enough hip capsule to create an adequate repair of the structure.

What may be desirable are bioinductive implants which include a synthetic and a biological layer and/or a bioinductive implant that is reinforced with a mechanically based structure.

Furthermore, during arthroscopic surgery, suture anchors are generally used to re-approximate soft tissue (such as a tendon or a ligament) back to a bone surface, thereby facilitating the natural healing process that restores the tendon/ligament-to-bone interface in a suture-based repair. The tendon/ligament-to-bone healing involves the formation of new tissue which mineralizes over time in gradient from soft tissue to bone, creating the transition zone that functions to transfer the mechanical forces from one tissue type to the other. Both calcium and phosphate ions are required for mineralization and have been reported to enhance mineralization in tissues, such as those found in the tendon/ligament-to-bone interface. Accordingly, it is desirable to increase the amount of calcium and/or phosphate ions available to a site of soft tissue-to-bone repair to promote the reattachment of the soft tissue to the bone.

### BRIEF SUMMARY

In accordance with aspects of the disclosure, a tissue repair implant and/or scaffold is provided that combines the benefits of a bioinductive implant, which biologically augments a repaired tendon and improves healing, with an implant that provides mechanical augmentation for added strength.

An example repair implant includes a sheet-like first component and a second component with a first side surface of the second component disposed on the first component. The first component includes a biological layer. The second component includes a synthetic material and a plurality of pores.

In addition or alternatively, the biological layer is bioabsorbable.

In addition or alternatively, the repair implant further includes a low molecular weight collagen disposed within the pores of the second component.

In addition or alternatively, the repair implant further includes a third component comprising a biological layer and positioned on a second side surface of the second component, the second side surface opposing the first side surface.

In addition or alternatively, the second component includes one or more strands forming a fabric.

In addition or alternatively, the repair implant further includes one or more loops extending generally orthogonal to a plane of the second component.

In addition or alternatively, the one or more loops are configured to attach to the sheet-like first component.

In addition or alternatively, a surface of the second component in contact with the first component is chemically modified to covalently bond the first component and the second component.

In addition or alternatively, a surface of the second component in contact with the first component is chemically modified to ionically bond the first component and the second component.

In addition or alternatively, the second component comprises one or more strands forming a ripstop pattern.

In addition or alternatively, the sheet-like first component includes collagen.

In addition or alternatively, the second component includes a generally solid layer and the plurality of pores are mechanically introduced.

In addition or alternatively, the synthetic material is non-resorbable polyester.

In addition or alternatively, the synthetic material is ultra-high molecular weight (UHMW) polyethylene.

In addition or alternatively, the first component and the second component are stitched together.

Another exemplary repair implant includes a sheet-like first component and a second component having a first side surface disposed on the first component. The sheet-like first component includes a biological layer. The second component includes a lattice structure.

In addition or alternatively, the lattice structure is formed at least in part from a bioabsorbable material.

In addition or alternatively, the lattice structure is formed at least in part from a non-bioabsorbable material.

In addition or alternatively, the lattice structure is formed at least in part form a non-bioabsorbable material.

In addition or alternatively, the repair implant further includes a third component comprising a biological layer and positioned on a second side surface of the second component, the second side surface opposing the first side surface.

In addition or alternatively, the biological layer is bioabsorbable.

In addition or alternatively, the second component is mechanically coupled to the first component.

In addition or alternatively, the biological layer includes collagen.

In addition or alternatively, the bioabsorbable material of the lattice structure includes collagen.

In addition or alternatively, the bioabsorbable material of the lattice structure includes polylactic acid.

In addition or alternatively, the second component is 3D printed.

In addition or alternatively, the second component is injection molded.

In addition or alternatively, the lattice structure includes a material formed into a pattern of interconnected diamonds.

In addition or alternatively, the lattice structure further includes one or more border lines extending about a perimeter of the pattern of interconnected diamonds.

In addition or alternatively, the lattice structure includes a plurality of cross-hatched cords.

In addition or alternatively, the lattice structure includes a first plurality of zig-zag cords and a second plurality of zig-zag cords. The second plurality of zig-zag cords are at least partially overlapping the first plurality of zig-zag cords.

In addition or alternatively, the lattice structure includes a plurality of interconnected circles.

Another exemplary repair implant includes a sheet-like first component and a reinforcing strand interwoven into the first component. The first component includes a biological layer.

In addition or alternatively, the biological layer is bioabsorbable.

In addition or alternatively, the reinforcing strand is a different color than the first component.

In addition or alternatively, a stitching pattern of the reinforcing strand changes geometries a long a length and/or width of the repair implant.

In addition or alternatively, the reinforcing strand comprises a suture.

In addition or alternatively, the reinforcing strand is interwoven in a direction extending generally parallel to a longitudinal dimension of the sheet-like first component.

In addition or alternatively, the reinforcing strand is interwoven in a direction extending generally orthogonal to a longitudinal dimension of the sheet-like first component.

In addition or alternatively, the reinforcing strand is interwoven in a direction extending generally non-orthogonal to a longitudinal dimension of the sheet-like first component.

In addition or alternatively, the reinforcing strand is interwoven in two or more directions relative to a longitudinal dimension of the sheet-like first component.

In addition or alternatively, the reinforcing strand extends through a thickness of the sheet-like first component.

In addition or alternatively, the reinforcing strand extends partially through a thickness of the sheet-like first component.

In addition or alternatively, the repair implant further includes one or more loops within an edge of the sheet-like first component or external to the edge of the sheet-like component.

In addition or alternatively, the one or more loops are configured to support attachment of the repair implant to a native structure.

Another exemplary repair implant may comprise a plurality of high strength filament loops, each loop of the plurality of high strength filament loops including a first end point and a second end point. The plurality of high strength filament loops are overlaid at varying angles to one another.

In addition or alternatively, the first end point and the second end point of at least some loops of the plurality of high strength filament loops may extend from a first edge of the repair implant to a second edge of the repair implant.

In addition or alternatively, one or more of the first or second end points of at least one of the loops of the plurality of high strength filament loops may be positioned a distance inward from an outer edge of the repair implant.

In addition or alternatively, each loop of the plurality of high strength filament loops may be formed as a discrete loop.

In addition or alternatively, the plurality of high strength filament loops may be formed from a single monolithic filament.

In addition or alternatively, the plurality of high strength filament loops may be fused together at some intersections of overlapping loops.

Further described herein is a composition that delivers calcium and/or phosphate ions in combination with collagen which acts as a scaffold for new tissue formation and can be placed locally between soft tissue and bone to facilitate healing of the soft tissue-bone interface. The composition can be pre-dried into a thin sheet of material and delivered as a pre-formed matrix, or as an injectable gel or paste which sets in place to form the matrix between the soft tissue and bone. The compositions of this disclosure advantageously increase the production of repair tissue at the soft tissue bone interface and thereby increase the rate of regain of mechanical competence of the healing tissue.

Examples of the composition of this disclosure may include one or more of the following, in any suitable combination.

In examples, a composition for soft tissue-to-bone repair of this disclosure is made up of collagen, a calcium compound and a phosphate compound. The calcium compound and the phosphate compound are evenly distributed throughout the composition. The composition forms a biocompatible matrix for insertion at an interface between soft tissue and bone and provides a stable mechanical environment for promoting mineralization of the tissue and/or bone. In examples, the composition is in the form of an injectable gel or paste. In other examples, the composition is in the form of a dried sheet. In examples, the collagen is a solubilized, pepsin-treated collagen. In examples, the calcium compound is calcium sulfate. In examples, the phosphate compound is sodium phosphate. In examples, a concentration of the collagen in the composition is about 40-50 mg/ml. In examples, a concentration of calcium in the composition is about 5% by weight of the composition. In other examples, a concentration of calcium in the composition is about 10% by weight of the composition. In yet further examples, a concentration of calcium in the composition is about 50% by weight of the composition.

Examples of a method of making a composition for soft tissue-to-bone healing of this disclosure include preparing a first amount of a calcium compound and preparing a second amount of an aqueous solution of sodium phosphate. A third amount of a collagen solution is added to the second amount of the aqueous solution of sodium phosphate. The third amount of the collagen solution and the second amount of the aqueous solution of sodium phosphate is mixed to a pH of about 6 to 7, causing the collagen solution to precipitate to form a collagen suspension. The collagen suspension is centrifuged until a volume of the collagen suspension is about 5 ml. The collagen suspension is then homogenized. After cooling, the first amount of the calcium compound is then added to the homogenized collagen suspension and mixed to form the composition of this disclosure. The composition is in the form of an injectable gel or paste.

In further examples, the first amount of the calcium compound is 34 mg. In other examples, the first amount of the calcium compound is 86 mg. In yet further examples, the first amount of the calcium compound is 340 mg. In examples, the collagen in the collagen solution is a solubilized, pepsin-treated collagen. In examples, the second amount of the aqueous solution of sodium phosphate is about 2.84 ml. In examples, the third amount of the collagen solution is 35 ml. In examples, the method further includes loading the composition into a syringe for injection at a soft tissue-bone interface of a patient. In other examples, the method further includes freeze drying the composition into a sheet for insertion at a soft tissue-bone interface of a patient.

Examples of a method of attaching a soft tissue to a bone of this disclosure include performing a surgical repair at a soft tissue-bone interface site of a patient and injecting a gel or paste containing a composition at the interface site. The composition is made up of collagen, a calcium compound and a phosphate compound. The calcium compound and the phosphate compound are evenly distributed throughout the composition. In other examples, the method includes performing a surgical repair at a soft tissue-bone interface site of a patient and inserting a dried sheet containing a composition at the interface site. The composition is made up of collagen, a calcium compound and a phosphate compound. The calcium compound and the phosphate compound are evenly distributed throughout the composition.

An example method of repairing damaged tissue in a joint having a bursa comprises withdrawing bursal fluid from the bursa, securing a sheet-like implant over the damaged tissue, and adding the bursal fluid to the sheet-like implant.

In addition or alternatively, the damaged tissue is a tendon.

In addition or alternatively, the joint is a shoulder.

In addition or alternatively, the bursal fluid is added to the sheet-like implant prior to securing the sheet-like implant over the damaged tissue.

In addition or alternatively, the bursal fluid is added to the sheet-like implant after securing the sheet-like implant over the damaged tissue.

In addition or alternatively, the implant is dried and adding the bursal fluid to the implant includes rehydrating the dried implant in the bursal fluid before securing the implant over the damaged tissue.

In addition or alternatively, adding the bursal fluid to the implant includes injecting the bursal fluid under the implant after securing the implant over the damaged tissue.

In addition or alternatively, the implant is hydrated and adding the bursal fluid to the implant includes infusing the bursal fluid into the hydrated implant.

In addition or alternatively, the sheet-like implant comprises a first component comprising a biological layer and a second component comprising a synthetic material, a first side surface of the second component disposed on the first component.

In addition or alternatively, the synthetic material includes a plurality of pores.

In addition or alternatively, the first component includes collagen.

In addition or alternatively, the method further comprises a low molecular weight collagen disposed within the pores of the second component.

The above summary of some examples and embodiments is not intended to describe each disclosed embodiment or every implementation of the present disclosure. The Brief Description of the Drawings, and Detailed Description, which follow, more particularly exemplify these embodiments, but are also intended as exemplary and not limiting.

### BRIEF DESCRIPTION OF THE DRAWINGS

FIG. 1 is a schematic anterior perspective view of a portion of the human shoulder;

FIG. 2 is a simplified perspective view of the human rotator cuff and associated anatomical structure;

FIG. 3 is a schematic depiction of a full thickness tear in the supraspinatus tendon of the rotator cuff of FIG. 2;

FIG. 4 is an anterior view showing the upper torso of a patient with the left shoulder shown in cross-section;

FIG. 5 is an enlarged, cross-sectional view showing the left shoulder depicted in FIG. 4;

FIG. 6 is an enlarged schematic cross-sectional view of a shoulder showing partial thickness tears and an exemplary repair implant positioned thereon;

FIG. 7 is a schematic posterior perspective view of an illustrative superior capsular reconstruction;

FIG. 8 is a schematic anterior perspective view of a hip joint;

FIG. 9 is a schematic anterior perspective view of the hip joint including the hip joint capsule;

FIG. 10 is a schematic anterior perspective view of the hip joint including a plurality of ligaments;

FIG. 11 is a schematic lateral side view of the hip joint including a plurality of gluteal muscles;

FIG. 12 is a schematic perspective view of an illustrative repair implant;

FIG. 13A is a top view of an illustrative structural layer of an illustrative repair implant;

FIG. 13B is a bottom view of the illustrative structural layer of FIG. 13B;

FIG. 14 is a top view of another illustrative structural layer of an illustrative repair implant;

FIG. 15 is a top view of another illustrative structural layer of an illustrative repair implant;

FIG. 16 is a top view of another illustrative structural layer of an illustrative repair implant;

FIG. 17A is a top view of another illustrative structural layer of an illustrative repair implant;

FIG. 17B is a top view of another illustrative structural layer of an illustrative repair implant;

FIG. 18 is a top view of another illustrative structural layer of an illustrative repair implant;

FIG. 19A is a top view of another illustrative structural layer of an illustrative repair implant;

FIG. 19B is a top view of another illustrative structural layer of an illustrative repair implant;

FIG. 20 is a schematic perspective view of another illustrative repair implant;

FIG. 21 is a top view of another illustrative repair implant; and

FIG. 22 is a graph of the results of a mineralization assay of a composition of this disclosure using a Mc3T3 pre-osteoblast cell line.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

Definitions of certain terms are provided below and shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same or substantially the same function or result). In many instances, the terms “about” may include numbers that are rounded to the nearest significant figure. As used herein, the term “about” refers to a ±10% variation from the nominal value unless otherwise indicated or inferred. Other uses of the term “about” (i.e., in a context other than numeric values) may be assumed to have their ordinary and customary definition(s), as understood from and consistent with the context of the specification, unless otherwise specified.

The recitation of numerical ranges by endpoints includes all numbers within that range (e.g., 1 to 5 includes 1, 1.5, 2, 2.75, 3, 3.80, 4, and 5). It is specifically intended that the description include each and every individual subcombination of the members of such groups and ranges and any combination of the various endpoints of such groups or ranges.

As used in this specification and the appended claims, the singular forms “a,” “an,” and “the” include or otherwise refer to singular as well as plural referents, unless the content clearly dictates otherwise. As used in this specification and the appended claims, the term “or” is generally employed to include “and/or,” unless the content clearly dictates otherwise.

It should be understood that the expression “at least one of” includes individually each of the recited objects after the expression and the various combinations of two or more of the recited objects unless otherwise understood from the context and use. The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

The use of any and all examples, or exemplary language herein, for example, “such as,” “including,” or “for example,” is intended merely to illustrate better the present teachings and does not pose a limitation on the scope of the invention unless claimed. No language in the specification should be construed as indicating any non-claimed element as essential to the practice of the present teachings. As used herein, “patient” refers to a mammal, such as a human.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or able to be arranged with each other to form other additional embodiments or to complement and/or enrich the described embodiment(s), as would be understood by one of ordinary skill in the art.

As used herein, a “compound” refers to the compound itself and its pharmaceutically acceptable salts, hydrates and esters, and biologic variations, unless otherwise understood from the context of the description or expressly limited to one particular form of the compound, i.e., the compound itself, or a pharmaceutically acceptable salt, hydrate or ester thereof.

Where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein. It should be understood that the order of steps or order for performing certain actions is immaterial so long as the present teachings remain operable. Moreover, two or more steps or actions may be conducted simultaneously.

The disclosure generally relates to tissue repair implants. The tissue repair implants, or repair implant, may be a structural soft tissue repair implant for use with tissues such as, but not limited to, tendons and ligaments.

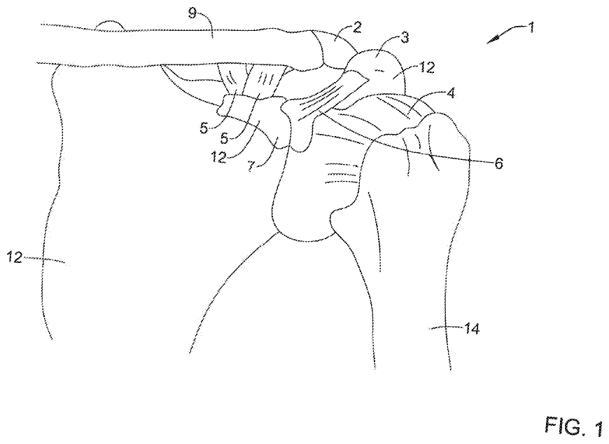

FIG. 1 is a schematic anterior perspective view of the shoulder 1 with some of the tendons, muscles, blood vessels, nerves, and bursae removed. The shoulder 1 may include additional structural components not explicitly shown and/or described. The present disclosure is not intended to provide complete anatomical details of the shoulder 1 but rather to provide an overview. Four joints make up the shoulder 1. These include the glenohumeral joint (not explicitly shown) where the ball of the humerus 14 fits into a shallow socket of the scapula 12, the acromioclavicular (AC) joint 2 where the clavicle 9 meets the acromion 3 (which is a part of the scapula 12), the sternoclavicular (SC) joint (not explicitly shown) which supports the connection of the arms and shoulders to the main skeleton at the chest, and the scapulothoracic joint (not explicitly shown) which is a fake joint where the scapula 12 slides across the rib cage (not explicitly shown).

The shoulder 1 further includes a plurality of ligaments which connect bones to bones. For example, the joint capsule 4 is a group of ligaments that connect the humerus 14 to the glenoid (not explicitly shown). The joint capsule 4 is a watertight sac that surrounds the glenohumeral joint and provides the main source of stability to the shoulder 1. For example, the joint capsule 4 helps keep the shoulder 1 from dislocating. The coracoclavicular ligaments 5 connect the clavicle 9 to the scapula 12. The AC joint 2 attaches the clavicle 9 to the scapula 12. Another ligament, the coracoacromial ligament 6 attaches the acromion 3 to the coracoid process 7 (which is a part of the scapula 12).

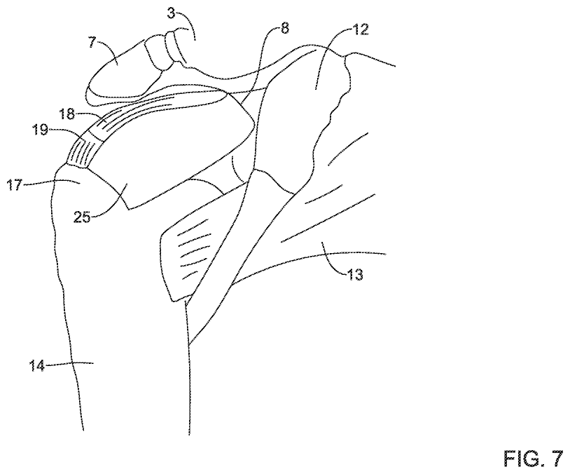

In some cases, rotator cuff tears may be irreparable. An alternative technique to repairing the rotator cuff directly is the superior capsular reconstruction (SCR) which utilizes the biological tissue scaffolds noted above, to stabilize the joint by direct attachment of the glenoid to the humeral head. For example, a scaffold, such as, but not limited to, the repair implants described herein, may be attached directly to the glendoid and the humeral head to help restore the position of the shoulder 1, as shown in more detail with respect to FIG. 7. The scaffold may be additionally attached to adjacent rotator cuff tissues.

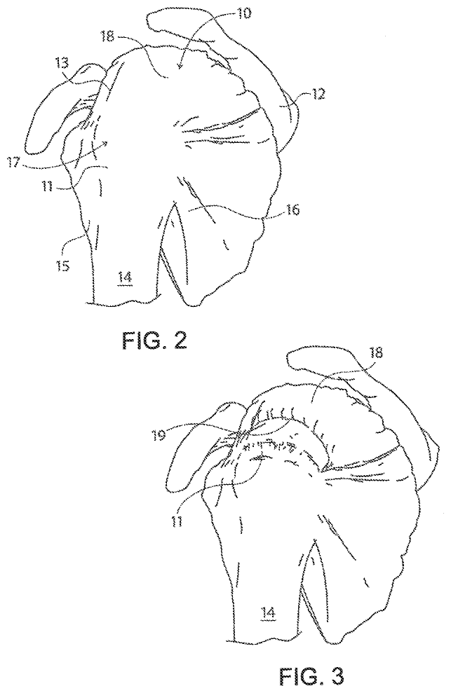

Referring additionally to FIG. 2 (and as disclosed by Ball et al. in U.S. Patent Publication No. 2008/0188936), the rotator cuff 10 is the complex of four muscles that arise from the scapula 12 and whose tendons blend in with the subjacent capsule 4 as they attach to the tuberosities of the humerus 14. The subscapularis 16 arises from the anterior aspect of the scapula 12 and attaches over much of the lesser tuberosity. The supraspinatus muscle 18 arises from the supraspinatus fossa of the posterior scapula 12, passes beneath the acromion 3 and the acromioclavicular joint 2, and attaches to the superior aspect of the greater tuberosity 11. The infraspinatus muscle 13 arises from the infraspinous fossa of the posterior scapula and attaches to the posterolateral aspect of the greater tuberosity 11. The teres minor 15 arises from the lower lateral aspect of the scapula 12 and attaches to the lower aspect of the greater tuberosity 11. Proper functioning of the rotator cuff 10 depends on the fundamental centering and stabilizing role of the humeral head 17 with respect to sliding action during anterior and lateral lifting and rotational movements of the arm.

The insertion of these tendons as a continuous cuff 10 around the humeral head 17 permits the cuff muscles to provide an infinite variety of moments to rotate the humerus 14 and to oppose unwanted components of the deltoid and pectoralis muscle forces. The insertion of the infraspinatus 13 overlaps that of the supraspinatus 18 to some extent. Each of the other tendons 16, 15 also interlaces its fibers to some extent with its neighbor's tendons. The tendons splay out and interdigitate to form a common continuous insertion on the humerus 14.

The rotator cuff muscles 10 are critical elements of this shoulder muscle balance equation. The human shoulder has no fixed axis. In a specified position, activation of a muscle creates a unique set of rotational moments. For example, the anterior deltoid can exert moments in forward elevation, internal rotation, and cross-body movement. If forward elevation is to occur without rotation, the cross-body and internal rotation moments of this muscle must be neutralized by other muscles, such as the posterior deltoid and infraspinatus. The timing and magnitude of these balancing muscle effects must be precisely coordinated to avoid unwanted directions of humeral motion. Thus the simplified view of muscles as isolated motors, or as members of force couples must give way to an understanding that all shoulder muscles function together in a precisely coordinated way (for example, opposing muscles canceling out undesired elements leaving only the net torque necessary to produce the desired action). Injury to any of these soft tissues can greatly inhibit ranges and types of motion of the arm.

The mechanics of the rotator cuff 10 are complex. The cuff muscles 10 rotate the humerus 14 with respect to the scapula 12, compress the humeral head 17 into the glenoid fossa providing a critical stabilizing mechanism to the shoulder (known as concavity compression), and provide muscular balance. The supraspinatus and infraspinatus provide 45 percent of abduction and 90 percent of external rotation strength. The supraspinatus and deltoid muscles are equally responsible for producing torque about the shoulder joint in the functional planes of motion.

With its complexity, range of motion and extensive use, a fairly common soft tissue injury is damage to the rotator cuff or rotator cuff tendons. Damage to the rotator cuff is a potentially serious medical condition that may occur during hyperextension, from an acute traumatic tear or from overuse of the joint. With its critical role in abduction, rotational strength and torque production, the most common injury associated with the rotator cuff region is a strain or tear involving the supraspinatus tendon. A tear in the supraspinatus tendon 19 is schematically depicted in FIG. 3. A tear at the insertion site of the tendon with the humerus, may result in the detachment of the tendon from the bone. This detachment may be partial or full, depending upon the severity of the injury. Additionally, the strain or tear can occur within the tendon itself. Injuries to the supraspinatus tendon 19 and recognized modalities for treatment are defined by the type and degree of tear. The first type of tear is a full thickness tear as also depicted in FIG. 3, which as the term indicates is a tear that extends through the thickness of the supraspinatus tendon regardless of the width of the tear. The second type of tear is a partial thickness tear which is further classified based on how much of the thickness is torn whether it is greater or less than 50% of the thickness.

The accepted treatment for a full thickness tear or a partial thickness tear greater than 50% includes reattaching the torn tendon to the humeral head using sutures. For the partial thickness tears greater than 50%, the tear is often completed to a full thickness tear by cutting the tendon prior to reattachment of the tendon. In treating a full thickness tear or partial thickness tear of greater than 50% after completing the tear by cutting the tendon, accepted practice also can include the placement of scaffolds and patches over the repaired tendon to shield the sutured or repaired tendon area from anatomical load during rehabilitation. For example, Wright Medical discloses that the GraftJacket® can be used to augment a suture repaired tendon in large and massive full thickness tears or smaller full-thickness tears in a shoulder having severely degenerated tissue. However, it is recognized that excessive shielding of the tendon from load can lead to atrophy and degeneration of the native tendon.

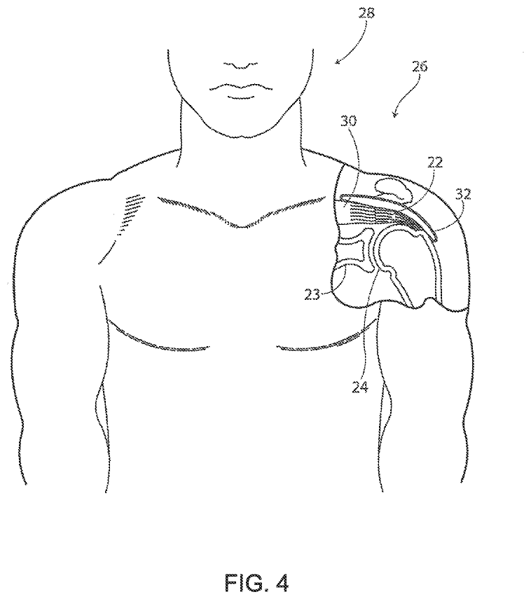

FIG. 4 is a stylized anterior view of a patient 28. For purposes of illustration, a shoulder 26 of patient 28 is shown in cross-section in FIG. 4. Shoulder 26 includes a humerus 24 and a scapula 23. The movement of humerus 24 relative to scapula 23 is controlled by the muscles of the rotator cuff as previously discussed with respect to FIG. 2. For purposes of illustration, only the supraspinatus 30 is shown in FIG. 4. With reference to FIG. 4, it will be appreciated that a distal tendon 22 of the supraspinatus 30 (hereinafter referred to as the supraspinatus tendon) meets humerus 24 at an insertion point 32.

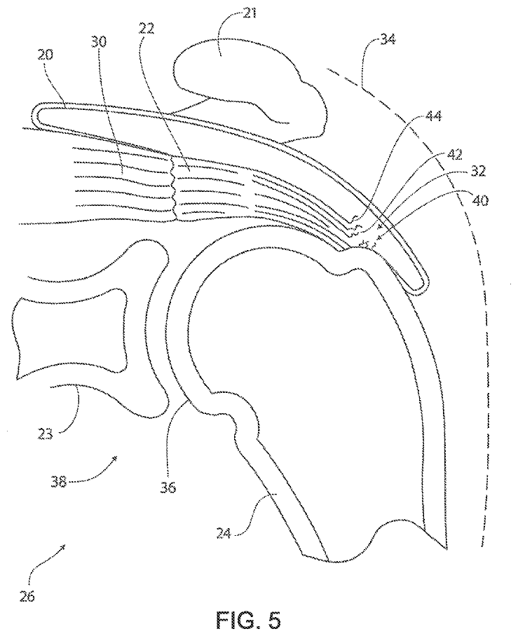

FIG. 5 is an enlarged cross-sectional view of shoulder 26 shown in the previous figure. In FIG. 5, a head 36 of humerus 24 is shown mating with a glenoid fossa of scapula 23 at a glenohumeral joint 38. The glenoid fossa comprises a shallow depression in scapula 23. A supraspinatus 30 and a deltoid 34 are also shown in FIG. 5. These muscles (along with others) control the movement of humerus 24 relative to scapula 23. A distal tendon 22 of supraspinatus 30 meets humerus 24 at an insertion point 32. In the embodiment of FIG. 5, tendon 22 includes a damaged portion 40 located near insertion point 32. Damaged portion 40 includes a tear 42 extending partially through tendon 22. Tear 42 may be referred to as a partial thickness tear. The depicted partial thickness tear is on the bursal side of the tendon; however, the tear can be on the opposite or articular side of the tendon or may include internal tears to the tendon not visible on either surface. Tendon 22 of FIG. 5 has become frayed. A number of loose tendon fibers 44 are visible in FIG. 5.

Scapula 23 includes an acromion 21. In FIG. 5, a subacromial bursa 20 is shown extending between acromion 21 of scapula 23 and head 36 of humerus 24. In FIG. 5, subacromial bursa 20 is shown overlaying supraspinatus 30. Subacromial bursa 20 is one of more than 150 bursae found the human body. Each bursa comprises a fluid filled sac. The presence of these bursae in the body reduces friction between bodily tissues.

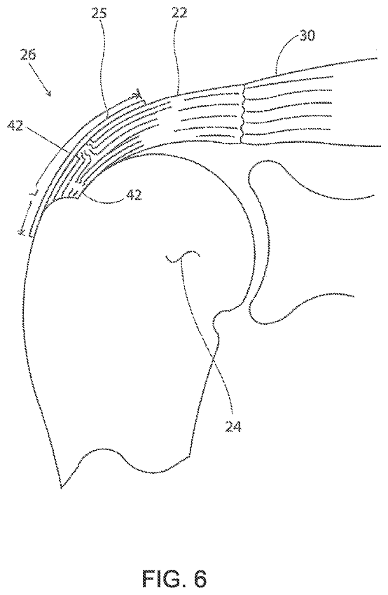

FIG. 6 is an additional cross-sectional view of shoulder 26 shown in the previous figure. In the embodiment of FIG. 6, a tissue repair implant 25 has been placed over the partial thickness tear 42. While the repair implant 25 has been described as being placed over a tendon, it should be understood that the repair implant 25 may be used to connect bone to bone. Further, while the tear 42 is illustrated as a partial thickness tear, it should be understood the implant 25 can also be used to repair a full thickness tear. For example, the implant 25 may be used to reinforce a repair that is fully approximated or to bridge between the tendon and the bone when the tendon cannot be approximated back to the bone. Other repair scenarios may include but are not limited to use with a full-thickness tear that has been repaired by re-approximating the torn tendon to the humeral head with sutures. In such an instance, the implant 25 may be placed on the bursal surface of the repaired tendon. In the illustrated embodiment, the repair implant 25 is placed on the bursal side of the tendon regardless of whether the tear is on the bursal side, articular side, or within the tendon. Further, the repair implant may overlay multiple tears, as an articular sided tear is also shown in FIG. 6. The implants disclosed herein may provide additional tensile strength while maintaining the ability of the implant 25, or portions of the implant 25, to sometimes be completely absorbed and remodeled by the body.

In addition to the rotator cuff, the repair implant 25 may be used with other body tissues, such as, but not limited to the superior capsule (e.g., as in a SCR), hip capsule, gluteus medius tendon, gluteus minimus tendon, Achilles tendon, etc., which may benefit from the use of a repair implant 25. FIG. 7 is schematic partial posterior perspective view of the shoulder 1 with a repair implant 25 being used in a superior capsular reconstruction. As described above, the repair implant 25 described herein, may be attached directly to the glenoid 8 and the humeral head 17 to help restore the position of the shoulder 1. The repair implant 25 may be additionally attached or sutured to (not explicitly shown) adjacent rotator cuff tissues, such as the supraspinatus muscles 18 and/or supraspinatus tendon 19. It should be noted that while not explicitly shown, the repair implant 25 is fixed to the glenoid 8 and the humeral head 17 using appropriate bone anchors and suturing.

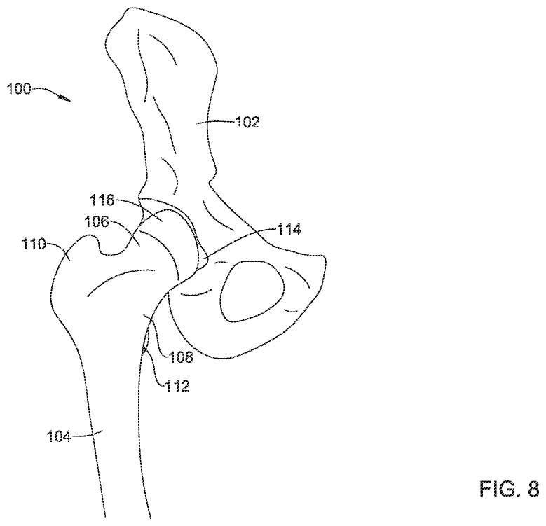

FIG. 8 is a schematic anterior perspective view of the hip joint 100 with the tendons, ligaments, muscles, blood vessels, nerves, and bursae removed. The hip joint 100, pelvis 102 and/or the femur 104 may include additional structural components not explicitly shown and/or described. The present disclosure is not intended to provide complete anatomical details of the hip joint 100 but rather to provide an overview. The hip joint 100 is a synovial ball and socket joint which couples the pelvis 102 and the femur 104. The hip joint 100 is very stable in contrast to the shoulder which is very mobile but not as stable as the hip joint 100. The proximal end of the femur 104 includes the femoral head 106, the femoral neck 108, the greater trochanter 110, and the lesser trochanter 112. The femoral head 106 is seated in the acetabulum 114 which is a circular depression in the pelvis 102. Both the socket portion (not explicitly shown) of the acetabulum 114 and the femoral head 106 include articular cartilage 116 disposed on a surface thereof. The articular cartilage 116 allows the bones 102, 104 of the joint 100 to slide against one another with no damage. Additionally the articular cartilage 116 absorbs shocks and provides a smooth surface to make motion easier. The ligamentum teres (not explicitly shown) attaches the femoral head 106 to the acetabulum 114. A small artery is within the ligamentum teres which provides some blood to the femoral head 106.

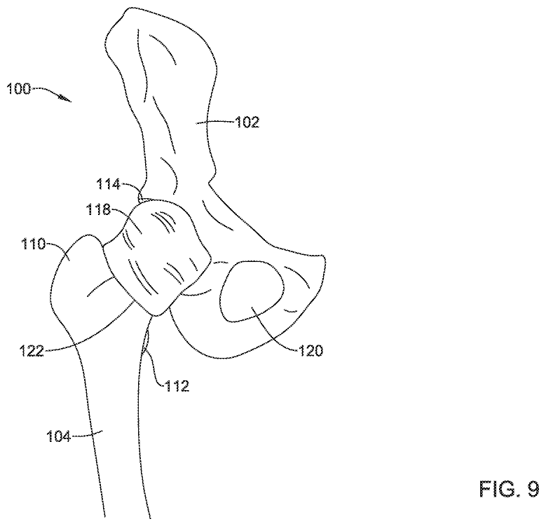

FIG. 9 is a schematic anterior perspective view of the hip joint 100 including the hip joint capsule 118. The joint capsule 118 is a water tight sac that surrounds the joint 100 and connects the pelvis 102 and the femur 104. The joint capsule 118 may have a thickness in the range of about 1.3 millimeters to about 7 millimeters. However, it is contemplated that the thickness of the joint capsule 118 may be greater in a diseased hip. The joint capsule 118 is attached to the pelvis 102 on the margins of the acetabulum 114, the transverse ligament (not explicitly shown) and lies over the acetabular notch (not explicitly shown), and the border margin of the obturator foramen 120. The hip joint capsule 118 is attached to the femur 104 anteriorly at the intertrochanteric line 122 which extends between the greater trochanter 110 and the lesser trochanter 112 and posteriorly at the intertrochanteric crest (not explicitly shown). The hip joint capsule 118 includes a group of strong ligaments that provide the main source of stability of the joint 100 and hold the femoral head 106 in place in the acetabulum 114. The hip joint capsule 118 is in part high strength due to the forces it withstands during various hip ranges of motion. The main blood supply for the femoral head 106 comes from blood vessels that travel underneath the hip joint capsule 118. The hip joint capsule 118 is routinely cut in order to perform arthroscopic hip surgery. At the end of hip arthroscopy, it is desirable to repair the hip joint capsule 118. When the hip joint capsule 118 is repaired, it is desired to ensure that the previously cut section re-heals. In some cases, the joint capsule tissue 118 may be substantially degenerated or there may not be enough hip joint capsule 118 to create an adequate repair of the structure. This may be due to partial capsulectomy (intentional or non-intentional), or a patient who desires to have more hip flexibility and repairing the native capsule would result in a hip with more difficult range of motion (for example, patients such as butterfly goalies or ballet dancers). It may be desirable to place the repair implant over the repaired capsule to protect the sutured or repaired area from excessive load during rehabilitation.

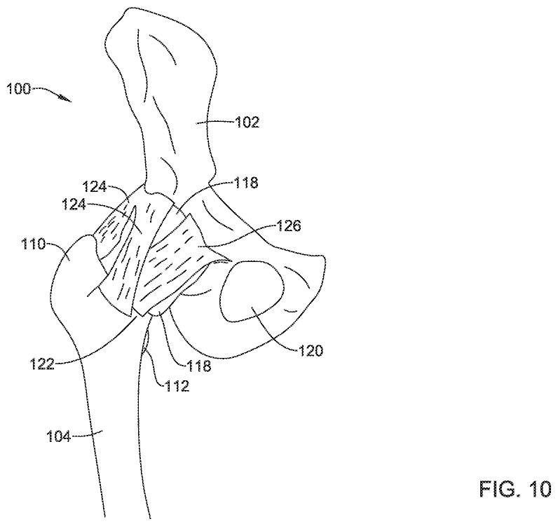

FIG. 10 is a schematic anterior perspective view of the hip joint 100 including a plurality of ligaments which reinforce the hip joint capsule 118. These ligaments include the iliofemoral ligament 124, the pubofemoral ligament 126, and the ischiofemoral ligament (not explicitly shown). The iliofemoral ligament 124 may be generally “Y” shaped. The iliofemoral ligament 124 attaches to the pelvis 102 at between the anterior inferior iliac spine (not explicitly shown) and the margin of the acetabulum 114 and to the femur 104 at the intertrochanteric line 122. The pubofemoral ligament 126 between the pelvis 102 at the iliopubic eminence of the pelvis 102 and the femur 104. The ischiofemoral ligament is posteriorly located and extends between the ischium (not explicitly shown) of the pelvis 102 and the greater trochanter 110 of the femur 104.

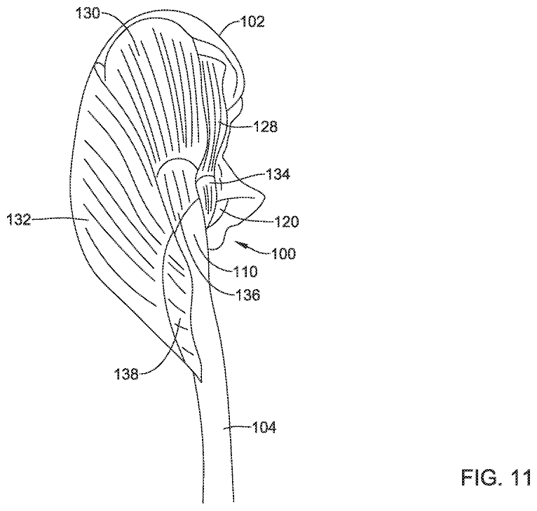

FIG. 11 is a schematic lateral side view of the hip joint 100 including a plurality of gluteal muscles. These muscles include the gluteus minimus 128, the gluteus medius 130, and the gluteus maximus 132. The gluteus minimus 128, gluteus medius 130, and gluteus maximus 132 may allow for extension of the leg and abduction of the leg. In some cases, partial or full thickness gluteal tears may occur in the tendons 134, 136, 138 which connect the muscle 128, 130, 134 to the femur 104. These tears may ultimately result in hip abduction weakness and abnormal gait, sometimes called the Trendelenburg gait. In some cases, the gluteal tendon tears are caused by attritional tendinopathy, which may be analogous to rotator cuff tears. In some cases, these tears can be treated with non-operative procedures, including but not limited to, activity modification, anti-inflammatory treatment, physical therapy, etc. Other hip pain may also be attributed to the gluteal tendons. For example, recalcitrant lateral hip pain, also known as greater trochanteric pain syndrome, may have a lesion of the gluteus medius and minimus tendons. These tears and lesions of the hip abductor tendons may be endoscopically repaired. However, failure rates of the repair may be as high as 35%. The repair implant can be placed over the repaired gluteal tendon to protect the sutured or repaired area from excessive load during rehabilitation.

The hip includes additional muscles, tendons, ligaments, blood vessels, nerves, and bursae that are not explicitly shown. For example, the hip includes the following muscles which are not explicitly shown: iliotibial band, adductor muscles, the iliopsoas, the rectus femoris, the sartorius, external rotators, and the hamstring muscles. These muscles in combination with the gluteal muscles 128, 130, 132 allow for abduction, adduction, flexion, extension, medial rotation, and lateral rotation of the leg. Abduction, adduction, flexion, and extension can be combined to produce circumduction.

In some embodiments, a repair implant is engineered to provide a combination of structural features, properties and functions that may be used to treat partial or full thickness tears of the rotator cuff and/or the gluteal tendons 134, 136, 138 and/or the repair the hip joint capsule 118. While the repair implant is described with respect to specific conditions and scenarios of the shoulder and hip, it should be understood that the repair implants described herein may be used in other conditions or scenarios where it is desirable to encourage tissue ingrowth while also providing additional mechanical properties such as, but not limited to strength and stiffness. These features, properties, and functions may include: rapid deployment and fixation by arthroscopic means that complement current procedures; tensile properties that result in desired sharing of anatomical load between the implant and native tendon during rehabilitation; selected porosity and longitudinal pathways for tissue in-growth; sufficient cyclic straining of the implant, having new tissue in-growth; induction of a healing response; and, in some cases, the repair implant is bioabsorbable or otherwise absorbable to provide transfer of additional load to native tendon over time.

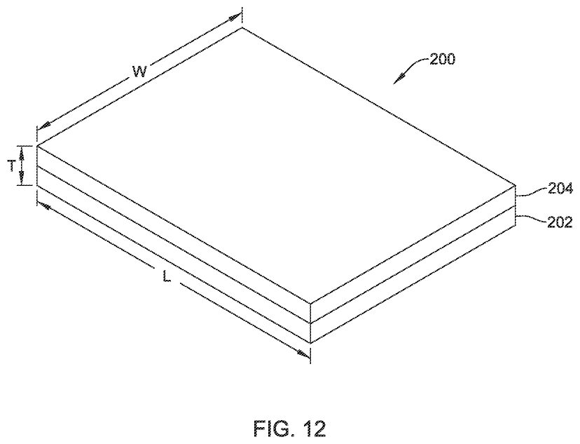

FIG. 12 is a perspective, schematic view of an illustrative tissue repair implant 200. While the repair implant 200 is illustrated as having a generally rectangular configuration, other shapes can be used, as desired. The repair implant 200 may be a multi-layer hybrid scaffold including a first or biological layer 202 and a second or structural layer 204. While the repair implant 200 is illustrated as having two layers, it is contemplated that the repair implant 200 may include any number of layers desired, such as, but not limited to, one, two, three, four, or more. Generally, the structural layer 204 has mechanical properties, such as strength, stiffness, creep, suture pull-out, etc., to support the load on the repair implant 200 upon initial implantation, while the biological layer 202 (e.g., collagen) provides rapid tissue ingrowth. The sheet-like structure 200 is defined by a longitudinal dimension L, a lateral dimension W and a thickness T. In some embodiments, lateral and longitudinal dimensions of the repair implant 200 may range from about 20 millimeters (mm) to 50 mm in the lateral direction W and 25 mm to 50 mm in the longitudinal direction L. The thickness T of the sheet-like structure may be about 0.5 mm to 5 mm when dehydrated. It is contemplated that the thickness of the implant 200 may be thicker, in the range of about 1 mm to 10 mm, when hydrated. Upon implantation, the longitudinal dimension L may extend generally in, or parallel to, the load bearing direction of the tendon. For example, in the embodiment shown in FIG. 6, the longitudinal direction L follows the supraspinatus tendon from its origin in the supraspinatus muscle down to the area of attachment on the humerus. As is well understood in the art, loading of the tendon is in this general direction upon contraction of the supraspinatus muscle.

While some previous implants may encourage rapid tissue ingrowth (e.g., blood vessels and fibroblasts), the previous implants may not provide any additional strength to the damaged tendon until new tissue is induced. In some instances, it may be desirable to provide a repair implant 200 which provides immediate mechanical strength to the tendon and also induces a healing response. This may be accomplished with a layered or composited implant 200. The implant 200 may include a first layer or component 202 having a first set of properties and a second layer or component 204 having a second set of properties. In some instances, the first layer 202 may be a bioinductive implant or component including a biological component such as, but not limited to, collagen, and the second layer 204 may be a higher-strength component formed from a synthetic material or a natural material structured for higher-strength. While FIG. 12 illustrates a single biological layer 202, it is contemplated that a second biological layer 202 may be positioned on the opposing side of the structural layer 204 such that the structural layer 204 is positioned between (e.g., sandwiched between) two biological layers 202. The two or more layers 202, 204 may be of similar thickness, or varying thicknesses, as desired.

The layer 202, 204 may be attached or coupled using a variety of techniques including, but not limited to mechanical coupling (e.g., suturing, weaving) and/or chemical coupling. In some cases, the layers 202, 204 may be attached to each other using medical grade fibers in stitching pattern to stitch the layers together. Alternatively or additionally, in some instances, the biological component 202 and the higher-strength component 204 may be formed as a laminated structure. For example, the biological component 202 and the higher-strength component 204 may be formed as discrete layers, as shown in FIG. 12. In other instances, the implant 200 may include a transition region between the layers 202, 204, such that the biological component 202 and higher strength component 204 are blended in the transition region. In yet other embodiments, the biological component 202 and the higher-strength component 204 may be an integrated composite (e.g., a single layer having both biological and synthetic aspects). For example, the higher-strength component 204 may be dispersed throughout the biological component 202. The biological component 202 and the higher-strength component 204 may have a different tensile modulus and a different tensile strength from one another. For example, the biological component 202 may have properties which encourage tissue ingrowth, or a healing response, while the higher-strength component 204 may have properties which provide immediate mechanical strength, as will be discussed in more detail below.

The biological component 202 may be a reconstituted collagen manufactured from highly purified type 1 collagen from bovine tendons. However, other sources of collagen may be used as well. The collagen fibers may be processed such that the biological component 202 extends into and/or surrounds the structural layer 204. In some embodiments, the biological layer 202 may have a lattice structure that can be 3D printed in a flat 2-dimensional pattern or a 3-dimensional pattern depending on the 3D printing technology utilized, as will be described in more detail herein. While 3D printing is one illustrative example, other suitable manufacturing techniques may be used as desired. For example, the lattice structure and/or other structures described herein may be formed by molding which may be punched, woven, etc. to form the desired arrangement. The biological layer 202 may be porous or included pathways to encourage tissue in-growth. In some embodiments, the sheet-like structure of the biological component 202 comprises a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. The porosity and tissue in-growth allows for new collagen to integrate with collagen of the native tendon for functional load carrying.

It will be appreciated that sheet-like structure may comprise various pore defining structures without deviating from the spirit and scope of the present description. In some embodiments, the sheet-like structure has a pore size in the range of about 20 to about 400 microns. In some embodiments the pore size is in the range of about 100 microns to about 300 microns, and in some embodiments it is about 150 to about 200 microns. The porosity may be about 30% to about 90%, or it may be within the range of at least about 50% to about 80%. In some instances, the biological component may have a dry density in the range of 0.2 grams per cubic centimeter (g/cm³) to 0.4 g/cm³. Examples of pore defining structures are discussed in more detail below for specific embodiments, but may include, but not be limited to open cell foam structures, mesh structures, micromachined layered structures and structures comprising a plurality of fibers. In some embodiments, the fibers may be interlinked with one another. Various processes may be used to interlink the fibers with one another. Examples of processes that may be suitable in some applications include weaving, knitting, crocheting, and braiding.

It is contemplated that the initial tensile modulus of the biological component 202 may be less than the tensile modulus of the supraspinatus tendon which is in the range of 50 MPa to 150 MPa. In some cases, some gluteal ligaments and/or tendons may have a tensile modulus in the range of about 4 MPa to about 22 MPa. Other gluteal ligaments and/or tendons may have a tensile modulus of about 24 MPa to about 25 MPa. The iliofemoral ligament 124 may have a tensile modulus in the range of about 76 MPa to about 286 MPa at 80% strain or about 1 to about 3.3 MPa at 0% strain. For example, the biological component 202 may be designed to have a tensile modulus in the range of 5 MPa to 50 MPa. In some embodiments, the tensile modulus may be approximately 10 MPa.

It is desirable in some situations to generate as much tissue as possible within anatomical constraints. In some cases where a tendon is degenerated or partially torn, tendon loads are relatively low during early weeks of rehabilitation. For example, in the shoulder, the load may be about 100 N. The strain in the tendon due to the load during rehabilitation can be about 2%. In some of these cases, the biological component 202 can be designed to have an initial ultimate tensile strength of at least about 2 MPa. The tensile strength may be designed to be no more than about 50 MPa and no less than about 5 MPa with a failure load of approximately 50 N to 100 N. The compressive modulus may be designed to be at least about 0.2 MPa. Similarly, the suture pull-out strength may be relatively low. For example, the suture pull-out strength may be in the range of 5 N to 15 N. It should be understood that the biological layer 202 can be designed to have initial ultimate tensile strengths, tensile strengths, failure loads, compressive moduli, suture pull-out strengths, etc. for use in regions of the body outside of the shoulder

The biological component 202 may be configured to allow loading and retention of biologic growth factors. The biological component 202 and/or the growth factors may be configured to controllably release the growth factors. The biological component 202 may be configured to allow transmission of body fluid to remove any degradation by-products in conjunction with a potential elution profile of biologics. The biological component 202 may also include platelet rich plasma at the time of implant or other biologic factor to promote healing and tissue formation.

The second, or higher-strength, component 204 may be generally stronger than the first component 202, having both a higher initial tensile strength and a higher initial tensile modulus than the first component 202. For example, the second component 204 may have a tensile strength approximately equal to the tensile strength of the supraspinatus tendon, which may be in the range of 20 MPa to 30 MPa. In other uses, such as, but not limited to the hip capsule, gluteal tendons, Achilles tendon, etc., the second component 204 may be designed to have a tensile strength approximately equal to the tendon at the implant location. The tensile strength of the second component 204 may be four to five times greater than the tensile strength of the biological component. In some cases, the second component 204 may have a failure load in the range of about 200 N or greater, 300 N or greater, 400 N or greater, 500 N or greater.

To accomplish the desired degree of load sharing, the initial tensile modulus of the second component 204 should be in the same general range as the tensile modulus of the tendon. For example, for use in the rotator cuff, the second component 204 may have a tensile modulus in the range of 50 MPa to 150 MPa. This may allow the initial load on the implant 200 to be in the range of 50% or more. It is contemplated that when used to repair the rotator cuff, the implant 200 may need to carry loads in the range of 20 N to 80 N during rehabilitation. Rehabilitation loads for a hip capsule repair may be similar to that of a rotator cuff repair. It is contemplated that a repair implant for use in some repair scenarios (such as, but not limited to, SCR) will need to carry loads beyond the rehabilitation period and will require greater failure loads, such as a failure load in the range of about 200 N or greater, 300 N or greater, 400 N or greater, 500 N or greater.

The suture pull-out strength of the second component 204 may be higher than the suture pull-out strength of the first component 202. For example, the suture pull-out strength of all of the sutures combined needs to be sufficient to support the load of a worst case scenario (e.g. attachment of the implant to both bone and tendon). As noted above for use in the rotator cuff, the implant 200 may need to carry loads of up to 80 N during the early rehabilitation period. The implant 200 may need to carry loads of 140 N or greater during normal use. If four sutures are used to affix the implant 200, each suture would need a pull out strength in the range of about 35 N or greater. In some cases, the sutures may have a pull out strength of 100 N or more. It is contemplated that the suture pull-out strength may be increased by chemical bonding through cross-linking and/or suturing the first and second components 202, 204 together around the perimeter thereof.

The load requirements for the repair implant 200 may vary depending on the tendon, ligament, or other soft tissue to be repaired. For example, the load requirements associated with “irreparable” rotator cuff tears (e.g., when the tendon is so far retracted that it cannot be reattached to the humeral head that the implant has to be bridge the gap between the tendon and the humeral head) or when a SCR is needed may be greater than a partial tear repair. It is contemplated that when the repair implant 200 is used to bridge the gap between the tendon and the humeral head or a SCR is required (these are just some examples), the repair implant 200 may carry the load indefinitely. For example, in these instances, the repair implant 200 does not transfer the load to the repaired tendon.

In one example, the second component 204 may be designed to provide stress protection until the repaired rotator cuff tendon reattaches to the humeral head. In other examples, the second component 204 may be designed to provide stress protection until the hip capsule, gluteal tendons, Achilles tendon, or other damaged tissue is healed. This may occur over a time period of 3 to 6 months. Thus, the second component 204 may maintain its strength for at least 3 to 6 months, and in some instances, longer and then begins to biodegrade. The second component 204 may comprise one or more bioabsorbable materials. Examples of bioabsorbable materials that may be suitable in some applications include those in the following list, which is not exhaustive: polylactide (PLA), poly-L-lactide (PLLA), poly-D-lactide (PDLA), polyglycolide (PGA), PGA/PLA blends, polydioxanone, polycaprolactone, polygluconate, polylactic acid-polyethylene oxide copolymers, modified cellulose, collagen, poly(hydroxybutyrate), polyanhydride, polyphosphoester; poly(amino acids), poly(alphahydroxy acid) or related copolymers materials. In some embodiments, the hydrothermal transition temperature may be selected to provide a desired absorption time. It is contemplated that the second component 204 may be absorbed more slowly than the biological component 202. For example, the second component 204 may be completely absorbed in about one year. This is just an example. In some instances, the second component 204 may be formed of a material that is not bio absorbable. For example, the second component 204 may be formed from a non-resorbable polyester, a high-tenacity polyester, polytetrafluoroethylene (PTFE), polyether ether ketone (PEEK), nylon, or ultra-high molecular weight (UHMW) polyethylene. The material of the second component 204 selected should be highly pure and have excellent biocompatibility in order to avoid an adverse inflammatory response. In yet other embodiments, the second component 204 may be formed from a combination or mixture of absorbable and non-absorbable materials. In some cases, a surface of the structural layer 204 may be chemically modified and a linking compound applied thereto such that the structural layer 204 is covalently or ionically bound to the biological layer 202.

The surface treatment may vary depending on the material forming the second component 204. For example, UHMW polyethylene may be pre-treated with air plasma to form unstable hydroperoxides on the surface there of. The surface may then be modified with acrylic acid and/or itatonic acid. The second component 204 may then be covalently bonded to between amino groups on the biological component 202 and carboxyl groups on the modified surface of the second component 204 in the presence of water-soluble carbodiimide/hydroxysuccinimide cross-linking system. Alternatively, UHMW polyethylene may be pre-treated with ammonia plasma. The pre-treated UHMW polyethylene may be reacted with dimethyl suberimidate. Next, the modified UHMW polyethylene may be reacted with a dispersion of collagen that has been buffered from a pH of about 3.5 to about 9 which causes collagen to precipitate. The modified UHMW polyethylene may be left in the collagen solution to allow the collagen to react with the modified UHMW polyethylene. In yet another example, polyester (PET) may be surface modified with sodium borohydride to reduce the carbonyl groups to alcohols. Next, a transesterification reaction may be performed with phenyl carbamate. The surface modified PET may then be reacted with dimethyl suberimidate. Next, the modified PET may be reacted with a dispersion of collagen that has been buffered from a pH of about 3.5 to about 9 which causes collagen to precipitate. The modified PET may be left in the collagen solution to allow the collagen to react with the modified PET. It is further contemplated that a coating, such as, but not limited to a collagen coating, to aid biological tissue attachment and integration may be applied to the surfaces of the second component 204. In yet another example, cross-linking of the second component 204 to the biological layer 202 may be done with formaldehyde.

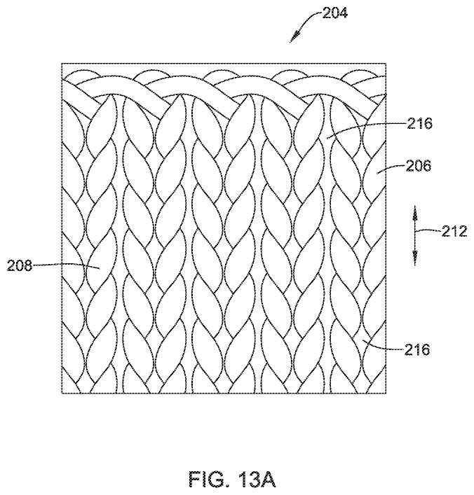

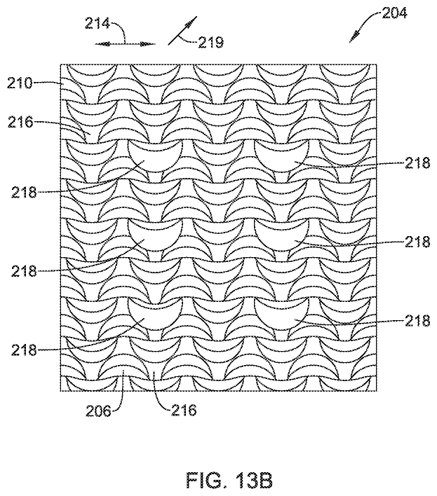

The structural layer 204 may include pores and/or pathways to allow for tissue ingrowth. FIG. 13A is a front view of an illustrative knit structural layer 204 and FIG. 13B is a back view of the illustrative knit structural layer 204 of FIG. 13A. The structural layer 204 illustrated in FIGS. 13A and 13B may be formed by knitting one or more filaments or strands 206 into a weft knit fabric. In a weft knit fabric, a single yarn may form one loop traveling in a weft direction 214. In some cases, the knit “stitch” may be selected to provide loops 208, 210 including a plurality of openings 216. In some cases, the loops 208, 210 may extend in different directions. For example, when viewing the front (FIG. 13A) of the structural layer 204, the loops 208 extend parallel to a first direction 212. When viewing the back (FIG. 13B) of the structural layer 204, the loops 210 extend parallel to a second direction 214 which is generally perpendicular to the first direction 212. Additional loops 218 may be provided which extend in a third direction 219 (e.g., generally orthogonal to the plane of the structural layer 204). These loops 218 may provide for adhesion points for the biological layer 202 to attach to. This may help prevent or minimize delamination of the structural layer 204 from the biological layer 202. It is contemplated that other knitting stitches or weaving patterns may be used to form the structural layer 204. For example, a ripstop pattern either at the edges or throughout the entire structural layer 204 (e.g., perpendicular strands, herringbone pattern, etc.) may be used to prevent unravelling if the repair implant 200 is cut. Further, a ripstop pattern may provide fixation points that may minimize the suture pulling through the edge of the repair implant 200.

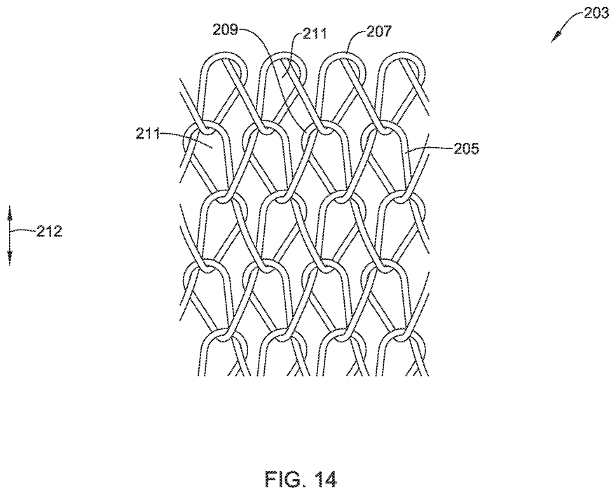

FIG. 14 is a front view of another illustrative knit structural layer 203. The structural layer 203 illustrated in FIG. 14 may be formed by knitting one or more filaments or strands 205 into a warp knit fabric. In some cases, the knit “stitch” may be selected to provide loops 207, 209 extending in a generally vertical (e.g., warp) direction 212 and including a plurality of openings 211. In some cases, the loops 207, 209 may be angled in different directions (e.g., zig-zag). Additional loops (not explicitly shown) may be provided which extend in a third direction (e.g., generally orthogonal to the plane of the structural layer 204). These loops may provide for adhesion points for the biological layer 202 to attach to. This may help prevent or minimize delamination of the structural layer 204 from the biological layer 202. It is contemplated that other knitting stitches or weaving patterns may be used to form the structural layer 204. For example, a ripstop pattern (e.g., perpendicular strands, herringbone pattern, etc.) may be used to prevent unravelling is the repair implant 200 is cut. Further, a ripstop pattern may provide fixation points that may minimize comb-out from the repair implant 200.

It is contemplated that other methods for forming a porous structure or fabric may also be used. For example, two or more filaments or strands may be woven together. It is contemplated that the size of the openings may be varied by varying how tightly the strands are woven. In another example, the structural layer may be formed using non-woven or non-knitted techniques. In such an example, short, or staple, fibers may be used to form the base structure. The base structure may be some degree randomly oriented with fibers aligning mostly in a first or “Y” direction, and some fibers extending in a second or “X” direction and some extending in a third or “Z” direction. In some cases, the alignment of the fibers can be broken up by puncturing the fabric with hooked needles and retracting the needles to bring some fibers through to the back side. The porosity of such a non-woven fabric can be manipulated by changing the fiber density. In some cases, the porosity can be in the 90% or greater range.

In some cases, knitted, crocheted, woven, and/or non-woven fabrics may be formed using a mixture of techniques (e.g., non-homogenous). For example, the border and/or one or more edges may be formed from a different knit stitch, weave, etc. as the center of the component 204. In some cases, a knit scaffold may have a woven border. This is just one example. One skilled in the art will recognize that there are any number of possible combinations. It is further contemplated that the biological component 202 may be formed using any of the knitted, woven, and/or non-woven techniques described herein.

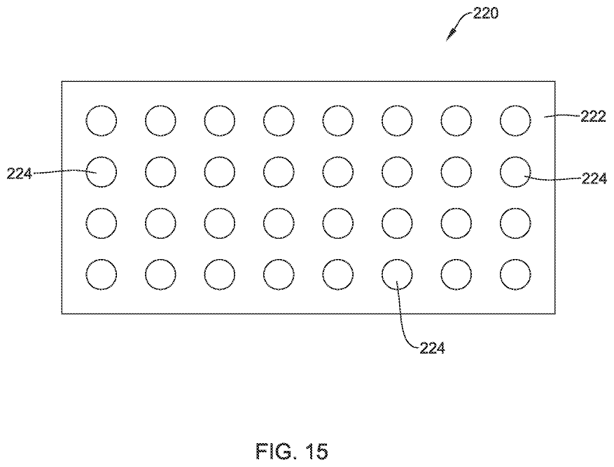

It is further contemplated that other structures or configurations may be used to introduce porosity into the structural layer 204, as desired. FIG. 15 illustrates a schematic top view of a structural layer 220 having a different structure from the structural layer 204. The structural layer 220 may be used in the repair implant 200 in place of, or in addition to the structural layer 204. The structural layer 220 may include a generally solid synthetic layer 222 with one or more mechanically introduced pores 224. For example, the generally solid synthetic layer 222 may be a polymer sheet or dense fiber. The pores 224 may be cut, molded, punched, etc. into the synthetic layer 222. In some cases, the structural layer 220 may be formed to have some flexibility. For example, the structural layer 220 may be a flexible construct of nitinol, other thin metallic structure (e.g., titanium alloys such as Ti6Al4V or stainless steel 300 series), a flexible polymer such as, but not limited to UHMWPE or polyglactin with a crimped pattern to allow for the expansion of the repair implant 200 in the hydrated state as well as compensating for pulling the biological layer 202 taut during placement of the repair implant 200. While the pores 224 are illustrated as having a generally equal or uniform spacing, it is contemplated that the pores 224 may be eccentrically spaced, if so desired. For example, the size, number, configuration, and/shape of the pores 224 may be varied to achieve the desired tissue ingrowth.

In some instances, a low molecular weight and/or low viscosity collagen may be applied within the openings 216 or pores 224 of the structural layer 204, 220. The presence of the collagen within the structural layer 204, 220 may further facilitate tissue ingrowth. The biological layer 202 including a higher molecular weight collagen may then be deposited on the structural layer 204, 220.

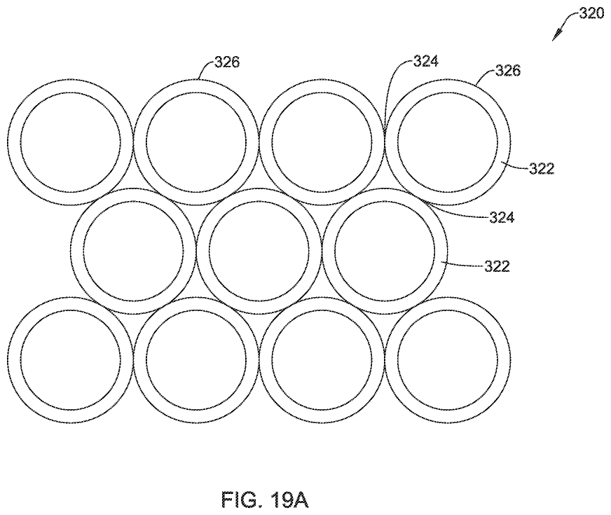

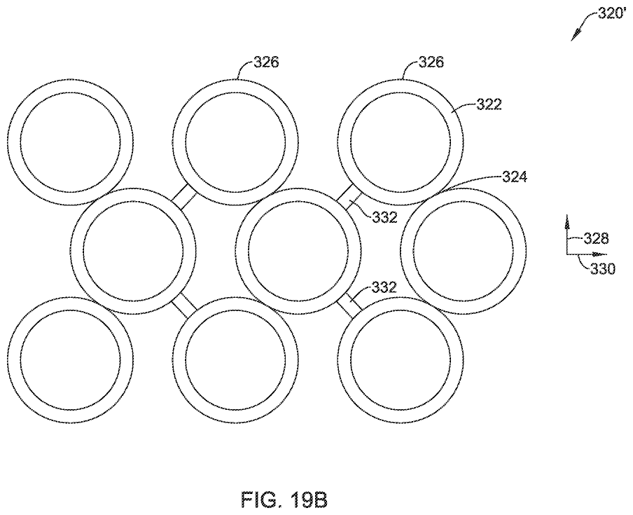

In some embodiments, the structural layer 204 of the repair implant 200 may be replaced with a collagen layer that includes a lattice structure designed to have the desired mechanical properties, such as strength, stiffness, creep, suture pull-out, etc., to support the load on the repair implant 200 upon initial implantation. A lattice structure may be a patterned, symmetrical, or asymmetrical distribution of collagen threads or filaments. The lattice structurer can include a single layer or multiple layers of threads or filaments, as desired. In one example, two to three (or more) biological layers 202 that are all type 1 collagen may be laminated together. One of those layers (e.g., replacing the structural layer 204) may be a structural lattice made from a dense collagen thread or filament that could be 3D printed in a variety of patterns to fine-tune structural performance. The other layer(s) (e.g., the biological layer 202) of 80% porous collagen fibers would be applied to this structural lattice layer on either one or both sides for the initial tissue in-growth. This may provide additional tensile strength to the repair implant 200 while maintaining its ability to completely absorb and be remodeled by the body. Additionally the structural lattice layer may be designed to have a specific elastic modulus that best supports the remodeling of the newly formed tendon-like tissue. Further, in some instances, the structural lattice layer may be designed to have an upper elastic limit (e.g., a degree of stretchability) where after the repair implant 200 has stretched a certain amount the fibers are designed in a way to now be straight structural cords that can provide a high degree of strength and stiffness that can help the newly formed tissue not experience an over-stretch and possible damage (such as, but not limited to, the embodiments illustrated and described without respect to FIG. 18, FIG. 19A, and FIG. 19B). For example, the structure fibers may be oriented generally oblique to the direction of the load. Thus, when a load is applied and the lattice is elongated, the fibers may straighten out (e.g., in the direction of the load) such that they are structurally bearing the force. Thus, the material will be stiffer and not stretch beyond a desired threshold.

It is contemplated that a structural lattice layer may be 3D printed in a flat 2-dimensional pattern or a 3-dimensional pattern depending on the 3D printing technology utilized. For example, extruder based 3D printing may be best for 2-dimensional pattern creation for a structural lattice layer that will later be laminated with highly porous type 1 collagen for tissue in-growth. While 3D printing is one illustrative example, other suitable manufacturing methods may be used, as desired.

In one example, type 1 collagen is extruded in a thick paste-like form out of a small nozzle that controls the diameter of the filament or cord being deposited onto the flat dry platform. The extruder nozzle is computer numerically controlled to extrude in a 2-dimensional pattern. The printer can then adjust the nozzle height off the platform and print additional layers on top of previously printed layers to create a 3-dimensional structure, as will be described in more detail herein. The liquid or paste-like collagen may then be freeze dried or other acceptable drying methods (such as, but not limited to, air drying or oven drying) to maintain the printed structure so that it can be transferred to another process where low density, high porosity collagen is applied to this structural lattice by spray.

In another example, type 1 collagen is extruded in a thick paste-like form out of a small nozzle that controls the diameter of the filament or cord being deposited in a gel-like vat that has sufficient density to keep the extruded material suspended in space and not sink to the bottom of the vat. Suspending the printed material within gel allows geometries to be created in 3D space that don't required a support structure to be printed along with the desired structure. The support structures are temporary and removed in post-processing, and often limits the kinds of geometries that can be 3D printed.

In yet another example, stereolithography (SLA) may be used. In SLA, the type 1 collagen is in liquid form and the energy from a laser cures or cross-links a specific point. The laser point moves to create a solid pattern. This may involve post-processing where un-cured liquid is removed from the solid printed structure and finally the solid printed structure needs to undergo an additional curing stage to bring it to full strength.

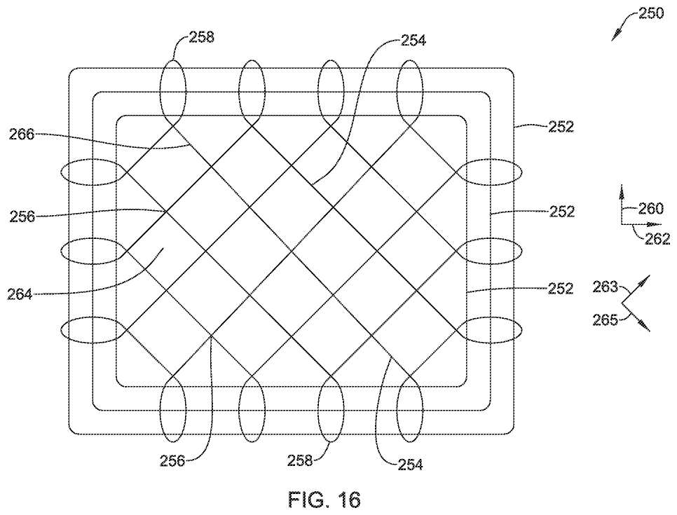

FIG. 16 illustrates a schematic top view of an illustrative structural layer having a lattice structure 250 that may be formed with collagen but also enhances the structural properties of the repair implant 200. The lattice structure 250 may be formed from an extruded line of highly dense collagen that is in a thick gel-like state with the use of solvents as a suspension as it is extruded. The solvents evaporate and leave behind solid cords of collagen. The diameter of the cords may be customized for a specific target strength. The lattice structure 250 may include one or more boundary or perimeter lines 252. The boundary lines 252 may be formed a single, spiraling cord or as a plurality of interconnected cords, as desired. It is further contemplated that the lattice structure 250 may include any number of boundary lines 252 desired, such as, but not limited to, one, two, three, four, or more. It is contemplated that the boundary lines 252 may be designed or structure for specific applications (e.g., specific anatomical locations) to reinforce the areas where tendon anchors and/or bone anchors are secured, which may provide increased tear-out resistance.

A crisscrossing diamond pattern 254 may be created within the boundary lines 252 for the lattice structure 250. In some cases, the diamond pattern 254 may be formed as a single, unbroken continuous line. In other cases, the diamond pattern 254 be formed from a plurality of interconnected line or cord 266. The diamond pattern 254 may further include a plurality of loops 258 which couple the diamond pattern 254 to the boundary lines 252. When the diamond pattern 254 is formed from an unbroken continuous line, the plurality of loops 258 may be a part of the continuous line. When extruding the paste, the extruder can apply pressure at specific times to press the lines together to form adhesion points 256. The diamond pattern 254 may have some flexibility in orthogonal directions 260, 262 and rigidity in diagonal directions 263, 265. The pattern or arrangement of the lattice structure 250 may be varied to adjust the strength of the repair implant 200 to suit a particular anatomy. For example, the diamond pattern 254 need not be symmetrical or uniformly arranged. It is contemplated that a thickness of the cord 266, spacing of the cord 266 (e.g., density) etc. are just some of the parameters that can be adjusted to achieve a lattice structure 250 of the desired structural properties. For example, that the spacing of the diamond pattern 254 may be varied to form smaller or larger openings. The slope of the cord 266 forming the diamond pattern 254 may also be varied to vary to the angle at the intersection points 256. For example the intersection points 256 may include non-orthogonal angles. These are just some examples of how the strength or other structural features of the lattice structure 250 can be manipulated.

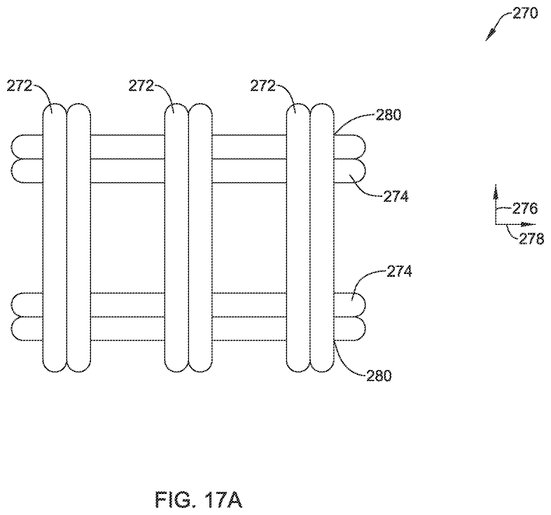

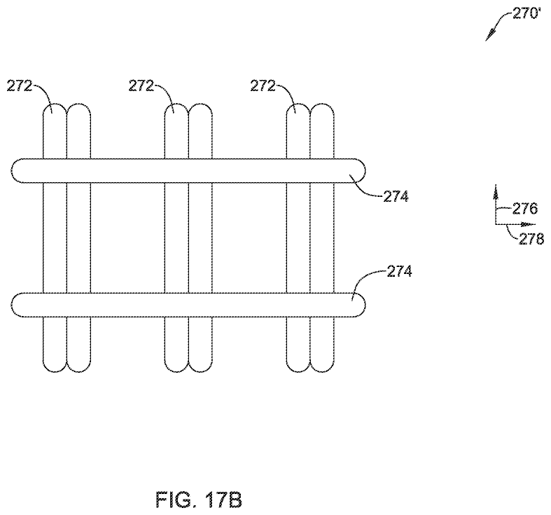

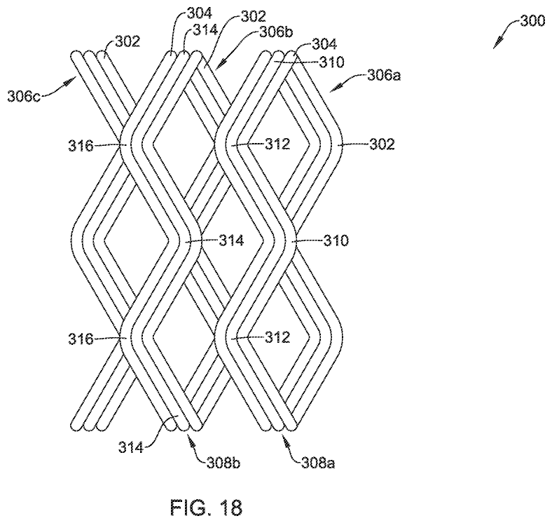

FIG. 17A illustrates a schematic top view of an illustrative structural layer having a lattice structure 270 that may be formed with collagen but also enhances the structural properties of the repair implant 200. The lattice structure 270 may be formed from an extruded line of highly dense collagen that is in a thick gel-like state with the use of solvents as a suspension as it is extruded. The solvents evaporate and leave behind solid cords of collagen. The diameter of the cords may be customized for a specific target strength. The lattice structure 270 may be formed by a plurality of cross-hatched cords 272, 274. A first plurality of cords 272 may extend in a first direction 276 and a second plurality of cords 274 may extend in a second direction 278 generally orthogonal to the first direction 276. It is contemplated that the cords 272, 274 may be aligned to extend in a particular orientation when the repair implant 200 is implanted in the body. For example, when used to repair a rotator cuff, one set of cords may extend in a medial to lateral direction while the other set of cords may extend in an anterior to posterior direction (when looking at the rotator cuff). However, it should be understood that the implantation orientation of the cords 272, 274 may be varied based on the anatomy being repaired.

When extruding the paste, the extruder can apply pressure at specific times to press the lines together to form adhesion points 280. In some cases, the cords 272, 274 may be manipulated as they are extruded to form a basket weave or generally woven configuration (e.g., in an over/under alternating arrangement).

In the illustrated embodiments of FIG. 17A, the cords 272, 274 may be uniformly arranged in both the first direction 276 and the second direction 278. However, this is not required. The cords 272, 274 may be arranged in any pattern or arrangement desired. In some cases, the lattice structure 270 may include a pattern in which there are more cords or fibers extruded in one direction than the other direction. For example, FIG. 17B illustrates an alternative lattice structure 270′ in which there are more cords 272 extending in the first direction 278 than there are cords 274 extending in the second direction 278. This particular configuration may provide a higher strength in the first direction 276 relative to the second direction 278. The pattern of the lattice structure 270 may be varied to adjust the strength of the repair implant 200 to suit a particular anatomy. For example, the arrangement of the cords 272, 274 may be asymmetrical. It is contemplated that a thickness of the cords 272, 274, spacing of the cords 272, 274 (e.g., density), number of cords 272, 274 are just some of the parameters that can be adjusted to achieve a lattice structure 270 of the desired structural properties. It is further contemplated that the cords 272, 274 may extend at non-orthogonal angles relative to one another. In some cases, one or more perimeter cords may also be provided.

FIG. 18 illustrates a schematic top view of an illustrative structural layer having a lattice structure 300 that may be formed with collagen but also enhances the structural properties of the repair implant 200. The lattice structure 300 may be formed from an extruded line of highly dense collagen that is in a thick gel-like state with the use of solvents as a suspension as it is extruded. The solvents evaporate and leave behind solid cords 302, 304 of collagen. The diameter of the cords 302, 304 may be customized for a specific target strength. The lattice structure 300 may have more depth than the structures of FIGS. 13, 14a, and 14B. For example, the lattice structure 300 may be formed by overlapping two patterns. A first set of cords 302 may be used to form the first or lower pattern. In the illustrated embodiments, a first group 306a of three cords 302 are extruded side by side in a zig-zag or undulating pattern. A second group 306b of three cords 302 are extruded side by side in a zig-zag or undulating pattern similar to the first group 306a and laterally spaced therefrom. A third group of three cords 302 are extruded side by side in a zig-zag or undulating pattern similar to the second group 306a and laterally spaced therefrom. Together, the groups 306a, 306b, 306c (collectively, 306) form the lower pattern. It is contemplated that the lower pattern may include any number of groups 306 desired, such as, but not limited to, one, two, three, four, or more. Further, each group 306 may include any number of cords 302 desired, such as, but not limited to, one, two, three, four, or more. It is contemplated that each group 306 need not have the same number or cords 302. While the groups 306 are illustrated as forming a zig-zag pattern, other patterns or configuration may be used as desired.

Once the lower pattern has been extruded, the upper pattern may be extruded thereupon. A second set of cords 304 may be used to form the second or upper pattern. In the illustrated embodiments, a first group 308a of three cords 304 are extruded side by side in a zig-zag or undulating pattern which overlays both a portion of the first group 306a and a portion of the second group 306b of the lower pattern. The first group 308a of the upper pattern may overlay a portion of the first group 306a of the lower pattern at one or more intersection points 310. Similarly, the first group 308a of the upper pattern may overlay a portion of the second group 306b of the lower pattern at one or more intersection points 312. A second group 308b of three cords 304 are extruded side by side in a zig-zag or undulating pattern similar to the first group 308a and laterally spaced therefrom. The second group 308b may overlay both a portion of the second group 306b and a portion of the third group 306c of the lower pattern. For example, the second group 308b of the upper pattern may overlay a portion of the second group 306b of the lower pattern at one or more intersection points 314. Similarly, the second group 308b of the upper pattern may overlay a portion of the third group 306c of the lower pattern at one or more intersection points 316. The upper pattern may be pressed into the lower pattern at each of the intersection points 310, 312, 314, 316 to from a union or bonded joint. When the upper pattern is disposed over the lower pattern, the lattice structure 300 may have a diamond pattern. Together, the groups 308a, 308b (collectively, 308) form the upper pattern. It is contemplated that the upper pattern may include any number of groups 308 desired, such as, but not limited to, one, two, three, four, or more. Further, each group 308 may include any number of cords 304 desired, such as, but not limited to, one, two, three, four, or more. It is contemplated that each group 308 need not have the same number or cords 304. While the groups 308 are illustrated as forming a zig zag pattern, other patterns or configuration may be used as desired.

The pattern of the lattice structure 300 may be varied to adjust the strength of the repair implant 200 to suit a particular anatomy. For example, the arrangement of the cords 302, 304 may be asymmetrical. It is contemplated that a thickness of the cords 302, 304 spacing of the cords 302, 304 or groups thereof 306, 308 (e.g., density), number of cords 302, 304, number of groups 306, 308 are just some of the parameters that can be adjusted to achieve a lattice structure 300 of the desired structural properties. In some cases, one or more perimeter cords may also be provided.

FIG. 19A illustrates a schematic top view of an illustrative structural layer having a lattice structure 320 that may be formed with collagen but also enhances the structural properties of the repair implant 200. The lattice structure 320 may be formed from an extruded line of highly dense collagen that is in a thick gel-like state with the use of solvents as a suspension as it is extruded. The solvents evaporate and leave behind solid cords 322 of collagen. The diameter of the cords 322 may be customized for a specific target strength. The lattice structure 320 may be formed by a plurality of circles 326 that are joined or bonded 324 in a side by side arrangement. The circular pattern may create uniform elasticity in all directions. It is contemplated that the spacing of the circles 326 may be changed or varied to manipulate the structural properties of the lattice structure 300. For example, the circles 326 may be positioned such that they are in contact with adjacent circles 326, as shown in FIG. 19A. In other embodiments, at least some of the circles 326 may be spaced from adjacent circles. FIG. 19B illustrates a lattice structure 320′ in which the circles 326 contact adjacent circles 326 in a first direction 328 but are laterally spaced from adjacent circles 326 in a second direction 330. It is contemplated that laterally spaced circles 326 may be bonded or connected by one or more linear cords 332. Increasing the spacing between the circles 326 may change the structural properties of the lattice structure 300′ as well as increasing an area for tissue ingrowth.

The pattern of the lattice structure 320, 320′ may be varied to adjust the strength of the repair implant 200 to suit a particular anatomy. For example, the arrangement of the linear cords 322 and/or the circles 326 may be asymmetrical. It is contemplated that a thickness of the cords 322, spacing of the cords 322 (e.g., density), number of cords 322, shape of the circles 326 (e.g., a shape other than circles) are just some of the parameters that can be adjusted to achieve a lattice structure 320 of the desired structural properties. In some cases, one or more perimeter cords may also be provided.

In some embodiments, the lattice structures 250, 270, 270′, 300, 320, 320′ may be formed from a material other than collagen. For example, any of the lattice structures 250, 270, 270′, 300, 320, 320′ may be formed as molded part resorbable material like PLA and applying highly porous collagen fiber network to one or both sides of the lattice structures 250, 270, 270′, 300, 320, 320′ to form one or more biological layers 202. It is contemplated that injection molding the lattice structures 250, 270, 270′, 300, 320, 320′ (and/or other structural layers described herein) may provide the advantage of a high degree of repeatability and control of the shape of the lattice and diameter of each cord and the strength of each union between cords. Further advantages may include, but are not limited to, a high degree of scalability and perhaps easier control during the manufacturing process of applying the highly porous collagen onto one or both sides of the structural lattice.

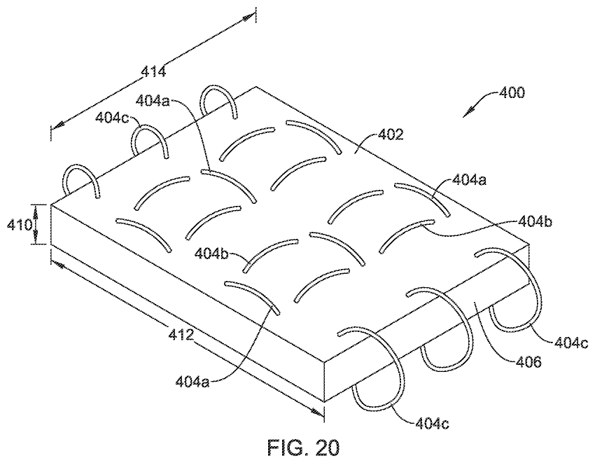

FIG. 20 is a perspective, schematic view of an illustrative tissue repair implant 400. The sheet-like structure 400 is defined by a longitudinal dimension 412, a lateral dimension 414 and a thickness 410. In some embodiments, lateral and longitudinal dimensions of the repair implant 400 may range from about 20 millimeters (mm) to 50 mm in the lateral direction 414 and 25 mm to 50 mm in the longitudinal direction 412. The thickness 410 of the sheet-like structure may be about 0.5 mm to 5 mm when dehydrated. It is contemplated that the thickness of the implant 400 may be thicker, in the range of about 1 mm to 10 mm, when hydrated. Upon implantation, the longitudinal dimension 412 may extend generally in, or parallel to, the load bearing direction of the tendon. While the repair implant 400 is illustrative as having a generally rectangular cross-sectional shape, other cross-sectional shapes may be used as desired, such as, but not limited to square, circular, oblong, polygonal, triangular, etc. It is further contemplated that the overall dimensions (e.g., longitudinal dimension 412, lateral dimension 414, and/or thickness 410) may be varied to suit a particular application.

The repair implant 400 may include a bioinductive scaffold 402 which may be similar in form and function to the biological layer 202 described herein. However, it is contemplated that the reinforcement structure described with respect to FIG. 20 may be used in combination with or in place of any of the structural layers descried herein. As described above, a bioinductive scaffold 402 by itself may not be able to withstand the loads exerted on it. Strands 404a, 404b, 404c (collectively, 404) may be woven into the bioinductive scaffold 402 as a reinforcement structure. The strands 404 may be formed from dermal tissue strands, sutures, or other biocompatible materials. If so provided, the sutures may be an absorbable type, a non-absorbable type, or combinations thereof. Other materials may be used in place of, or in addition to dermal tissue strands or sutures. Examples of bioabsorbable materials that may be suitable in some applications include those in the following list, which is not exhaustive: polyglactin, polylactide, poly-L-lactide (PLLA), poly-D-lactide (PDLA), polyglycolide (PGA), polydioxanone, polycaprolactone, polygluconate, polylactic acid-polyethylene oxide copolymers, modified cellulose, collagen, poly(hydroxybutyrate), polyanhydride, polyphosphoester; poly(amino acids), poly(alphahydroxy acid) or related copolymers materials. Examples of non-absorbable materials that may be suitable in some applications include those in the following list, which is not exhaustive: non-resorbable polyester or ultra-high molecular weight (UHMW) polyethylene.

The strands 404 may be interwoven into the bioinductive scaffold 402 in various weaving and/or stitching patterns. For example, any number of stitch configurations could be imagined that would reinforce the material (different numbers of rows, orientations, stitch patterns, crossing geometries, etc.) and provide the specific mechanical properties (e.g., tensile strength, etc.) to the bioinductive scaffold 402. In some cases, the strands 404 may have loops, a helical arrangement, a row of helical strands, zig-zag arrangement, or other sinusoidal patterns. The illustrated example is just one of many possible patterns. It is further contemplated that strands 404 of varying thickness may also be used. In some cases, a geometry of the strand 404 and/or the pattern of the stitch may vary over the width and/or length of the bioinductive scaffold 402. In some cases, the strands 404 may be provided as a preformed arrangement that can be attached to the bioinductive scaffold 402 by the clinician.

In an example, some strands 404a may be interwoven such that they extend generally parallel to the longitudinal dimension 412. The strands 404a may extend through the entire thickness 410 of the bioinductive scaffold 402 (e.g., such that strands 404a are visible on an opposing side of the bioinductive scaffold 402) or may extend partially through the thickness 410 of the bioinductive scaffold 402 (e.g., such that strands 404a are not visible on an opposing side of the bioinductive scaffold 402). It in contemplated that the strands 404a may be interwoven as a single row or a plurality of rows (as illustrated in FIG. 20). It is contemplated that the number of stiches per row may also vary. For example, if more than one row is provided, each row may have same number of stitches or a differing number of stitches, as desired. In some cases, a length (e.g., in the longitudinal dimension 412) of the stitch may be varied. It is contemplated that decreasing a length of the stitch while increasing the quantity of stitches may increase the strength of the repair implant 400. In some cases, the color of the strand 404 (e.g., the stitches) may be differentiated from the color of the bioinductive scaffold 402 in order to facilitate placement of the scaffold. For example, such color differentiation may provide a visualization aid for the surgeon.

Alternatively, or additionally, some strands 404b may be interwoven such that they extend generally parallel to the lateral dimension 414. The strands 404b may extend through the entire thickness 410 of the bioinductive scaffold 402 (e.g., such that strands 404b are visible on an opposing side of the bioinductive scaffold 402) or may extend partially through the thickness 410 of the bioinductive scaffold 402 (e.g., such that strands 404b are not visible on an opposing side of the bioinductive scaffold 402). It in contemplated that the strands 404b may be interwoven as a single row or a plurality of rows (as illustrated in FIG. 20). It is contemplated that the number of stiches per row may also vary. For example, if more than one row is provided, each row may have same number of stitches or a differing number of stitches, as desired. In some cases, a length (e.g., in the lateral dimension 414) of the stitch may be varied. It is contemplated that decreasing a length of the stitch while increasing the quantity of stitches may increase the strength of the repair implant 400.

While the strands 404a, 404b are described as extending generally orthogonal to one another (and parallel to the dimensions of the bioinductive scaffold 402) it is contemplated that other orientations may be used as well. For example, one or both of the strands 404a, 404b may extend at non-parallel and/or non-orthogonal angles to one another and/or relative to the dimensions of the bioinductive scaffold 402. It is further contemplated that strands may be interwoven in single orientation (parallel, orthogonal, non-orthogonal, or otherwise) relative to the dimensions of the bioinductive scaffold 402.

In some embodiments, some strands 404c may be positioned at the edges of the bioinductive scaffold 402. The strands 404c may form loops that are accessible from a point exterior to the bioinductive scaffold 402. Said differently the strands 404c may be within the edges 406 of the bioinductive scaffold 402 or external to the edges 406 such that the strands 404c may be used to support attachment to the native structure. In some cases, the strands 404 may also be used for delivery or manipulation of the bioinductive scaffold 402 for placement of the bioinductive scaffold 402. It is contemplated that the strands 404c may be positioned about an entire perimeter or border of the bioinductive scaffold 402 or only portions thereof, as desired. Further, there may be any number of strands 404c desired, such as but not limited to, one, two, three, four, or more.

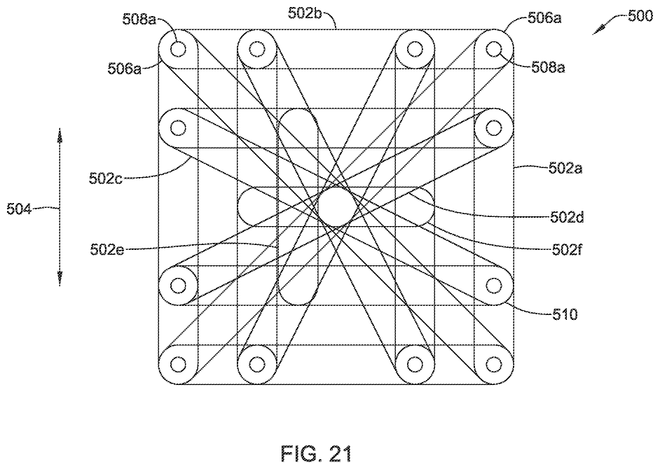

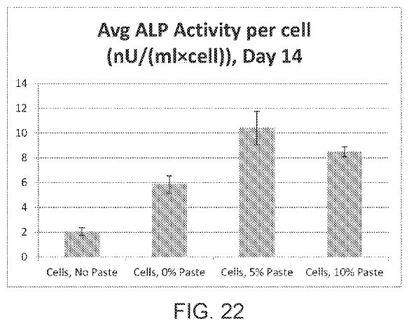

FIG. 21 is a top view of another illustrative tissue repair implant 500. In addition to use as a standalone implant, the repair implant 500 may be used as a reinforcing layer in a manner similar to the structural layers described herein. The repair implant 500 may be a porous structure including a plurality of overlapping high strength filament loops 502a, 502b, 502c, 502d, 502e, 502f (collectively, 502). It should be noted that for brevity every distinct loop is not individually identified in FIG. 21. Each loop 502 may be formed from a thread or filament. The loops 502 may be affixed to one another by fusing or bonding each intersection 510 of the filaments forming the loops 502. This may be done using, for example, heat or adhesives. However, it is contemplated that not every intersection be fused. Each loop 502 may be formed individually as a continuous loop and subsequently assembled into the repair implant 500. For example, rather than being a woven or knitted material, the loops 502 may be laid over each other in varying orientations (e.g., angles relative to one another) to form the repair implant 500. In some cases, as the loops 502 are positioned, the orientation of a subsequent loop 502 may be varied relative to the previously positioned loop 502, although this is not required. This configuration may carry tension between opposing points 506a, 506b (collectively 506) around the perimeter of the repair implant 500. It should be noted that each loop 500 includes opposing points; however, for brevity, on the points 506a, 506b on a single loop 502b have been specifically labeled. While not explicitly shown, in some cases, a loop 502 may be positioned so that it generally aligns with a previously laid loop (either directly on top of or having other loops 502 therebetween).

In some cases, suture anchors (not explicitly shown) may be positioned in the bone at the repair location with the suture anchors generally corresponding to the opposing points 506 of one or more loops 502. Sutures 508a, 508b may pass through the repair implant 500 at each of the points 506 (or a subset thereof) to secure the repair implant 500 within the body. If a suture happens to pass through the filament forming a loop 502, there may be very little elongation of the repair implant 500. In some cases, the sutures may be tied together to form a mattress stitch or they may be affixed directly to the repair implant 500. It is contemplated that the repair implant 500 may share the shear load between all of bone anchors

Some loops 502a may extend generally parallel to a length direction 504 of the implant 500. Other loops 502b may extend generally orthogonal to the length direction 504 of the implant 500. Yet other loops 502c, 502d may extend at non-orthogonal angle relative the length direction 504 of the implant 500. It is contemplated that the number of loops 502 extending in each direction may be varied to achieve desired structural properties. It is further contemplated that one or more loops 502e, 502f may not extend entirely across a periphery of the repair implant 500. For example, one or more ends of one or more loops 502e, 502f may be positioned a distance inward from a perimeter or outer edge of the repair implant 500. Including loops 502e, 502f that do not extend from edge to edge of the repair implant 500 may allow the repair implant 500 to be cut (e.g., to match a desired anatomy) while maintaining integrity for suture anchoring.

The repair implant 500 may include any number of loops 502 desired, such as, but not limited to two or more, five or more, ten or more, twenty or more, etc. It is contemplated that the physical properties of the repair implant 500 can be varied by varying the number of loops 502. For example, fewer loops 502 may result in a thinner, more porous repair implant 500. Increasing the number of loops 502 may increase a thickness (e.g., as loops 502 overlap) of the repair implant 500 and result in a generally less porous structure. While the loops 502 are described as being separate and discrete structures, in some cases, the repair implant 500 may be formed from one continues thread or filament that has been wound to achieve a similar structure to that illustrated in FIG. 21.

The overlapping loop structure of the repair implant 500 may improve the footprint of soft tissue compression against the bone by eliminating bias stretch that may be seen in a woven material. It is further contemplated sharing the shear and compression forces between several suture anchors the individual comb-out forces would be reduced thus reducing suture comb-out.

In other examples, a biologically active material may be added directly to any of the implants or scaffolds discussed herein, during implantation. In some embodiments, bursal fluid may be added to the implant. Bursal fluid is rich in biologically active agents including a population of stem cells, growth factors, and recruitment factors, and contributes to the native tendon-healing response. The fluid derived from the bursa may rival that of the stem cell population found in bone marrow as a biologically active pool of recruitment factors and healing potential.

During the implantation procedure of the any of the implants or scaffolds described herein, a bursectomy of the anatomical joint (e.g., shoulder joint) or other procedure may be performed to extract bursal fluid from the bursa of the anatomical joint. For example, bursal fluid, also known as synovial fluid, may be extracted from the bursal sac of the bursa of the anatomical joint with a syringe. The extracted bursal fluid (i.e., bursal fluid removed from the patient's body) may then be delivered back into the anatomical joint at the time of implanting the implant. In some instances, the extracted bursal fluid may be added to the implant or scaffold prior to securing the implant or scaffold over the damaged tissue (e.g., torn tendon) in the anatomical joint. For example, the extracted bursal fluid may be used to fill a hydrated implant or scaffold, which may be made of collagen, with the extracted bursal fluid using a large gauge needle syringe. As the bursal fluid comes from the patient, no treatment of the bursal fluid is needed prior to injecting the extracted bursal fluid into the implant or scaffold. Once the bursal fluid has been applied to the implant or scaffold, the implant or scaffold may be inserted into the joint space and secured to the damaged tissue. If desired, purified extracellular proteins may be added to the extracted bursal fluid and/or the implant during implantation of the implant.

In some embodiments, the implant may be hydrated in saline prior to delivery into the joint space. Another embodiment may involve hydrating a dehydrated implant in a bursal fluid (such as bursal fluid extracted from the patient during the medical procedure) or other derived biologically active slurry during the medical procedure prior to implantation of the implant. In some examples, the bursal fluid or other biologically active slurry may be mixed with saline. Hydrating the implant in bursal fluid with a suspended high concentration slurry of cellular matter may only require a few minutes to expect cell adhesion to the bursal side of the collagen implant.

In other instances, the extracted bursal fluid may be added to the implant after securing the implant over the damaged tissue (e.g., torn tendon). For instance, the extracted bursal fluid may be injected under the implant after the implant is in place and secured to the damaged tissue.

Alternatively, the extracted bursal fluid may be mixed with a coagulating material, such as a fibrin glue, and coated on both sides of the implant prior to securing the implant against the damaged tissue. In a still further embodiment, the extracted bursal fluid may be injected into a biocompatible sac that is disposed under the implant when implanted over the damaged tissue. The biocompatible sac may be permeable to allow the fluid to leach out onto the implant over a period of time (such as a period of days, weeks, or months) subsequent to the medical procedure.

An advantage provided by the addition of biological factors such as bursal fluid is an increased chance of healing for patients with a predisposition for poor healing, such as smokers or diabetics, by providing an additional form of recruitment. Furthermore, this modification may allow for a faster rate of healing compared to the unmodified implant alone.

According to aspects of the present detailed disclosure, methods of treating a tear or lesion in a tendon or ligament are also provided. In some methods, supraspinatus tendons, ligaments of the hip capsule, gluteal tendons, and/or Achilles tendons having complete tears, partial thickness tears of greater than 50% and/or partial thickness tears of less than 50%, or significant degeneration are treated. The treatment site may be first arthroscopically accessed in the area of the damaged tendon. However, in some cases, the tendon may be accessed using an open technique. A repair implant, such as previously described may be placed over a partial tear in a tendon. In some embodiments, the implant may be placed over a tendon having complete or partial tear(s), abrasions and/or inflammation. Left untreated, minor or partial tendon tears may progress into larger or full tears. According to aspects of the present disclosure, a complete or partial tear may be treated by protecting it with a repair implant as described above. Such treatment can promote healing and provide immediate strength to the tendon, as well as prevent more extensive damage from occurring to the tendon, thereby averting the need for a more involved surgical procedure.

For arthroscopic delivery of the repair implant, the implant may be configured to be collapsible so that it may be inserted into or mounted on a tubular member for arthroscopic insertion to the treatment site. For example, the implant and associated delivery device may be collapsed like an umbrella where the deployed delivery systems unfolds the pleats of the implant as mounted thereon to allow surface to surface engagement with the tendon without any substantial wrinkles. In some instances, once flat against the tendon, the repair implant may then be affixed using sutures or other suitable means such as staples such that the tensile properties will assure that the anatomical load will be shared because the native tendon and implant experience the same strain under load. In other instances, the repair implant may be affixed using bone screws and sutures, or other suitable means, such that the repair implant carries the entire load.

In summary, the repair implant may comprise an absorbable material, layers of absorbable materials, reinforced absorbable materials, and/or a combination of an absorbable material and a non-absorbable material. In some embodiments, the purpose of the implant is to protect an injured portion of a tendon during healing, provide an implant 200, 400 for new tissue growth, and/or temporarily share some of the tendon loads. The implant may induce additional tendon-like tissue formation, thereby adding strength and reducing pain, micro strains and inflammation. In some embodiments, the implant may substitute for tissues that cannot be repaired. When implanted, the implant 200, 400 may provide immediate strength to the tendon and transfer the load to the native tendon as the tendon heals and the implant 200, 400 biodegrades. In other embodiments, the repair implant 200, 400 may carry the entire load indefinitely. In some embodiments, organized collagen fibers are created that remodel to tendon-like tissue or neo-tendon with cell vitality and vascularity. Initial stiffness of the device may be less than that of the native tendon so as to not overload the fixation while tendon tissue is being generated.

Material(s) used in the implanted device should be able to withstand the compression and shear loads consistent with accepted post-surgical motions of the implantation location (e.g., shoulder, hip, knee, ankle, etc.). The perimeter of the device may have different mechanical properties than the interior of the device, such as for facilitating better retention of sutures, staples or other fastening mechanisms. The material(s) may be chosen to be compatible with visual, radiographic, magnetic, ultrasonic, or other common imaging techniques. The material(s) may be capable of absorbing and retaining growth factors with the possibility of hydrophilic coatings to promote retention of additives.

Further described herein are compositions which are intended to be used as a simple collagen matrix to deliver calcium ions (Ca⁺²) and/or phosphate ions (PO₄⁻³) to an interface between an injured tendon or ligament and bone to promote the healing of the attachment to the bone. Collagen is chosen as a delivery media as it is biocompatible, biodegradable and can be in the form of an injectable gel or paste that has the capability of setting up and forming a weak suspension or a dried sheet. This advantageously allows the Ca⁺² and/or PO₄⁻³ to be held in place at the interface and delivered locally while providing a transient collagen scaffold for new tissue formation.

In examples, the compositions of this disclosure comprise about 40-50 mg/ml collagen (+/−10%), such as fibrillar atelo collagen. In addition, anywhere from about 1-20% of the final percentage of a calcium compound, such as calcium sulfate (CaSO₄) (which translates into about 4-20 mg/ml of Ca⁺²), and a small amount of a phosphate compound, such as sodium phosphate (Na₂PO₄) (0.5-0.01 Molar final concentration range), is used during processing such that the calcium and phosphate compounds are evenly distributed throughout the composition. The dissolved hydrogen phosphate (HPO₄⁻²) will readily react with some of the dissolved Ca⁺² to form calcium phosphate until there are no free phosphate ions. Dissolved sodium chloride (NaCl) is also present in the mixture. It will be appreciated that other calcium compounds, such as calcium phosphate, calcium carbonate, calcium nitrate and/or calcium chloride, could also be used to form the compositions of this disclosure.

An example of a method for making an injectable gel form of the compositions of this disclosure will now be described.

Initially, the following amounts of CaSO₄ are transferred into two glass weighing funnels for the preparation of a first example of the composition containing a 5% concentration by weight of calcium ions and a second example of the composition containing a 50% concentration by weight of calcium ions. Optionally, a third example of the composition containing a 10% concentration by weight of calcium ions may be prepared:

| Final calcium content in | |
|---|---|
| composition | Mass of CaSO₄ |
| 5% wt | 34 mg |
| 10% wt | 86 mg |
| 50% wt | 340 mg |

Each of the funnels are placed into 250 ml beakers and covered with aluminum foil. The covered beakers are placed into an oven and heated at 250° C. for at least one hour to sterilize the calcium compound. However, other known methods of sterilization are contemplated by this disclosure.

Next, 0.25M of an aqueous solution of Na₂PO₄ is prepared by adding about 80 ml of deionized water to a 100 ml volumetric flask, followed by the addition of about 3.55 g of Na₂PO₄. The flask is placed on a stirrer plate until the Na₂PO₄ is dissolved. Deionized water is then added to the 100 ml mark of the flask. The Na₂PO₄ solution is then mixed and the volume is checked.

Next, a 50 ml centrifuge tube is prepared by transferring 35 ml of a collagen solution into the tube. In examples, the collagen solution may be a solubilized, pepsin-treated collagen (atelo collagen) of bovine origin. About 2.84 ml of the Na₂PO₄ solution is added to the centrifuge tube containing the 35 ml of collagen solution. The tubes are capped and mixed to a pH of about 6 to 7, causing the collagen to precipitate to form a fibrillar collagen suspension, which is white and opaque. About 3.8 ml of a pH buffer solution, such as 10× Dulbecco's phosphate buffered saline (DPBS), is then added to each of the collagen containing tubes, which are capped and mixed.

The collagen precipitate is centrifuged at 24000×g for about 4 minutes. After centrifugation, the volume of collagen is checked using the markings on the centrifuge tube. The volume at the end of centrifugation should be about 5 ml, which is equivalent to a target fibrillary collagen concentration of about 40±5 mg/ml. The collagen concentration is calculated according to the following equation:

$$C final = \frac{C start \times V start}{V final \times 0.95} \qquad ( Equation 1 )$$

where C is collagen concentration and V is volume. The factor 0.95 is for 95% yield in the process.

If the concentration is too low, the centrifuge tubes are placed back in the centrifuge and centrifuged for another 5 minutes. The target collagen concentration may also be adjusted using additional DPBS, if needed. The additional volume of DPBS to be added can be calculated using the following equation:

$$VDPBS = \frac{C start \times V start}{40 \times 0.95} - V current \qquad ( Equation 2 )$$

The calculated amount of 10× DPBS can be added to an empty 30 ml syringe and connected to the syringe containing the collagen concentration using a luer to luer connector and mixed by passing the collagen concentration from syringe to syringe at least 30 times. When the target collagen concentration is reached, the supernatant is decanted from the collagen suspension and the collagen suspension is homogenized using a high shear homogenizer. The 5 ml of collagen suspension is then transferred equally between two 10 ml syringes.

After the two beakers containing the respective 34 mg and 340 mg of CaSO₄ have been allowed to cool to room temperature, the two concentrations of CaSO₄ are transferred into two separate, clearly labeled, 10 ml syringes. One of the 10 ml syringes containing the collagen suspension is connected to the 10 ml syringe containing the 34 mg of CaSO₄ using a luer to luer connector and mixed by passing the collagen suspension from syringe to syringe at least 30 times. The other of the 10 ml syringes containing the collagen suspension is connected to the 10 ml syringe containing the 340 mg of CaSO₄ using a luer to luer connector and mixed by passing the precipitate from syringe to syringe at least 30 times. The two final injectable collagen/Na₂PO₄/Ca⁺² gel compositions (one having 5% calcium by weight and the other having 50% calcium by weight) are then loaded into syringes for injectable use on a patient.

An example of a method for making a dried sheet form of the compositions of this disclosure will now be described.

Initially, a suspension of collagen, Na₂PO₄ and CaSO₄ is produced as described above after adjusting the component input amounts to account for the increased volume and desired sheet size and density. This suspension is poured onto a flat mold with raised edges that will define the sheet dimensions (L×W), including the thickness of the desired final dried sheet. The mold with the suspension is then placed into a lyophilizer and freeze dried to produce the final dried sheet.

In examples, the compositions described herein can be used to augment a tendon-to-bone surgical repair, for example, a rotator cuff repair. For example, a surgical repair of the rotator cuff attachment to the underlying bone can be conducted per standard, suture anchor based techniques. During the re-attachment of the tendon to the bone, the injectable gel form of the composition can be introduced to the tendon-bone interface. For example, the injectable gel form of the composition can be injected via a syringe or similar device onto the bone surface at the location of the repair prior to the re-attachment of the tendon, but after suture anchor placement. In other examples, the dried sheet form of the compositions can be placed onto the bone surface at the location of the repair prior to the re-attachment of the tendon, and either before or after suture anchor placement. In other examples, after the surgical repair is completed, the injectable gel form of the compositions can be injected onto the tendon-bone interface by means of a syringe and a needle passed through the tendon tissue into the interphase between the tissues. In further examples, the injectable gel form of the compositions can be introduced into the central cannulation of an open architecture suture anchor via a modified cannulated suture anchor inserter with a syringe during anchor placement and prior to tissue re-approximation. Non-limiting examples of an open-architecture suture anchor can be found in U.S. Pat. No. 8,894,661 to Smith & Nephew, Inc. (Memphis, Tenn.), the entire contents of which are incorporated herein by reference. Non-limiting examples of a syringe can be found in International Publication No. WO 2017/213893 to Smith & Nephew, Inc. (Memphis, Tenn.), the entire contents of which are incorporated herein by reference.

In other examples, the compositions described herein can be used to augment a ligament repair, for examples, an anterior cruciate ligament (ACL) reconstruction. In examples, the surgical repair of the torn ACL is conducted per standard surgical techniques using a ligament graft. The dried sheet can then be placed at the graft-bone junction in the bone tunnels used for graft placement and fixation (either by suspensory fixation or with interference screws). In examples, the dried sheet can be wrapped around the end portions of the graft that will be placed into the bone tunnels during graft preparation prior to introduction of the graft in to the joint. Alternatively, the graft can be placed into the bone tunnels and then the injectable gel form of the composition can be injected around the graft in the bone tunnel prior to fixation via the use of a syringe. Example

The compositions of this disclosure were evaluated in a cell based system using the compositions of this disclosure having 0% wt CaSO₄, 5% wt CaSO₄ and 10% wt CaSO₄ respectively. MC3T3 pre-osteoblast cells were cultivated for 14 days using the three respective compositions. The cell based experiments demonstrated an increased expression of bone mineralization markers (e.g. alkaline phosphatase designated ALP) in cells cultured on the compositions as compared to collagen alone, as shown in FIG. 1. This demonstrated that the compositions described herein enhance new bone formation by MC3T3 osteoblast cells.

It is to be understood that even though numerous characteristics of various embodiments have been set forth in the foregoing description, together with details of the structure and function of various embodiments, this detailed description is illustrative only, and changes may be made in detail, especially in matters of structure and arrangements of parts illustrated by the various embodiments to the full extent indicated by the broad general meaning of the terms in which the appended claims are expressed. Scope of the disclosure is thus indicated by the appended claims, rather than by the foregoing description, and all changes that come within the meaning and range of equivalency of the claims are therefore intended to be embraced therein.

What is claimed:

1. A repair implant comprising:
a sheet-like first component comprising a biological layer; and
a second component, a first side surface of the second component disposed on the first component, the second component comprising a lattice structure.

2. The repair implant of claim 1, wherein the lattice structure is formed at least in part from a bioabsorbable material.

3. The repair implant of claim 2, wherein the lattice structure is formed at least in part from a non-bioabsorbable material.

4. The repair implant of claim 1, wherein the lattice structure is formed at least in part form a non-bioabsorbable material.

5. The repair implant of claim 1, further comprising a third component comprising a biological layer and positioned on a second side surface of the second component, the second side surface opposing the first side surface.

6. The repair implant of claim 5, wherein the biological layer is bioabsorbable.

7. The repair implant of claim 1, wherein the biological layer comprises collagen.

8. The repair implant of claim 1, wherein the bioabsorbable material of the lattice structure comprises collagen.

9. The repair implant of claim 1, wherein the bioabsorbable material of the lattice structure comprises polylactic acid.

10. The repair implant of claim 1, wherein the second component is 3D printed.

11. The repair implant of claim 1, wherein the second component is injection molded.

12. A repair implant comprising:
a sheet-like first component comprising a biological layer; and
a second component, a first side surface of the second component disposed on the first component, the second component comprising a synthetic material and including a plurality of pores.

13. The repair implant of claim 12, wherein the biological layer is bioabsorbable.

14. The repair implant of claim 13, further comprising a low molecular weight collagen disposed within the pores of the second component.

15. The repair implant of claim 12, further comprising a third component comprising a biological layer and positioned on a second side surface of the second component, the second side surface opposing the first side surface.

16. The repair implant of claim 12, wherein the second component comprises one or more strands forming a fabric.

17. The repair implant of claim 16, further comprising one or more loops extending generally orthogonal to a plane of the second component.

18. The repair implant of claim 17, wherein the one or more loops are configured to attach to the sheet-like first component.

19. The repair implant of claim 12, wherein a surface of the second component in contact with the first component is chemically modified to covalently bond the first component and the second component.

20. The repair implant of claim 12, wherein a surface of the second component in contact with the first component is chemically modified to ionically bond the first component and the second component.

21. The repair implant of claim 12, wherein the sheet-like first component comprises collagen.

22. The repair implant of claim 12, wherein the second component comprises a generally solid layer and the plurality of pores are mechanically introduced.

23. A repair implant comprising:
a sheet-like first component comprising a biological layer; and
a reinforcing strand interwoven into the first component.

24. The repair implant of claim 23, wherein the reinforcing strand is a different color than the first component.

25. The repair implant of any one of claims 33-35, wherein a stitching pattern of the reinforcing strand changes geometries along a length and/or width of the repair implant.

26. The repair implant of claim 23, wherein the reinforcing strand is interwoven in a direction extending generally parallel to a longitudinal dimension of the sheet-like first component.

27. The repair implant of claim 23, wherein the reinforcing strand is interwoven in a direction extending generally orthogonal to a longitudinal dimension of the sheet-like first component.

28. The repair implant of claim 23, wherein the reinforcing strand is interwoven in two or more directions relative to a longitudinal dimension of the sheet-like first component.

29. The repair implant of claim 23, further comprising one or more loops within an edge of the sheet-like first component or external to the edge of the sheet-like first component.

30. A method of repairing damaged tissue in a joint having a bursa, comprising:
withdrawing bursal fluid from the bursa;
securing a sheet-like implant over the damaged tissue; and
adding the bursal fluid to the sheet-like implant.

31. The method of claim 30, wherein the implant is dried and adding the bursal fluid to the implant includes rehydrating the dried implant in the bursal fluid before securing the implant over the damaged tissue.

32. The method of claim 30, wherein adding the bursal fluid to the implant includes injecting the bursal fluid under the implant after securing the implant over the damaged tissue.

33. The method of claim 30, wherein the implant is hydrated and adding the bursal fluid to the implant includes infusing the bursal fluid into the hydrated implant.
